(19) United States
(12) Patent Application Publication
(10) Pub. No.: US 2015/0238617 A1
(43) Pub. Date: Aug. 27, 2015

(54) SILK RESERVOIRS FOR SUSTAINED DELIVERY OF ANTI-CANCER AGENTS

(71) Applicant: TUFTS UNIVERSITY, Medford, MA (US)

(72) Inventors: David L. Kaplan, Concord, MA (US); Tuna Yucel, Medford, MA (US); Michael Lovett, Peabody, MA (US); Xiaoqin Wang, Winchester, MA (US)

(21) Appl. No.: 14/431,067

(22) PCT Filed: Oct. 11, 2013
(86) PCT No.: PCT/US13/64493

§ 371 Date: Mar. 25, 2015

Related U.S. Application Data
(60) Provisional application No. 61/712,571, filed on Oct. 11, 2012.

(51) Int. Cl. A61K 47/42; A61K 9/70; A61K 31/4402; A61K 9/00; A61K 31/4196

(52) U.S. Cl. CPC A61K 47/42; A61K 9/0024; A61K 31/4196; A61K 31/4402; A61K 9/70

(57) ABSTRACT

The present invention on directed to silk-based drug delivery compositions for sustained delivery of drugs, e.g., for cancer therapy, and methods of their use for treatment.

### CROSS REFERENCE TO RELATED APPLICATIONS

This application claims the benefit under 35 U.S.C. §119(e) of U.S. Provisional Application No. 61/712,571 filed Oct. 11, 2012, the content of which is incorporated herein by reference in its entirety.

### TECHNICAL FIELD

The present disclosure relates to silk compositions for sustained delivery of molecules, such as therapeutic agent(s), as well as methods of making and using the same. In one aspect the present disclosure relates to silk-based drug-delivery compositions for sustained delivery of cancer therapeutics and methods for treatment of cancer.

### BACKGROUND

Breast cancer, i.e., ductal and lobular carcinomas encompasses over 20% of all cancer cases in women worldwide. One of the treatment options for hormone receptor positive disease is the use of non-steroidal aromatase inhibitors, such as ARIMIDEX™. Anastrozole, the active ingredient of ARIMIDEX™, is a potent (1 mg administered orally, once a day) small molecule drug (C₁₇H₁₉N₅, m=293.4 Da) with moderate water solubility (0.5 mg/ml at 25° C.), moderate lipophilicity (log P(octanol/water)=1.58), and non-ionic character at neutral pH (pKa=1.4) [AstraZeneca Canada, Inc., ARIMIDEX™, Product Monograph, 2011]. However, there is growing concern about patient adherence to aromatase inhibitor therapy [Charlson, Proc. Am. Soc. Clin. Oncol. 2010; 28: 73s. Abstract 524] and large reported differences in patient self-report on adherence and actual medication delivery results even within the first year [Rey-Herin et al., Proc. Am. Soc. Clin. Oncol. 2010; 28: 102s Abstract 643]. One possible means to address adherence issues may be the sustained delivery of aromatase inhibitors, e.g., anastrozole. Clearly, the administration frequency for sustained release anastrozole formulations must be low enough, e.g., inter-administration durations of several months to render these formulations attractive in the clinic.

Accordingly, there is a need for improved pharmaceutical compositions lacking potentially inflammatory degradation byproducts that provide controlled, sustained delivery of therapeutic agent(s) which can improve compliance to breast cancer therapy.

### SUMMARY

The present disclosure provides silk-based drug delivery compositions that provide sustained delivery of therapeutic agent(s). In addition to fostering patient compliance, such silk-based drug delivery composition exhibit excellent biocompatibility and non-inflammatory degradation products, such as peptides and amino acids. Therefore, potential use of silk in sustained release pharmaceutical formulations as a carrier could minimize immune response, and enhance stability of an active ingredient as compared to other polymeric formulations with acidic degradation byproducts (e.g., PLGA). Silk compositions can be processed in completely aqueous based solvents. Accordingly, such silk-based drug delivery compositions avoid the use of hazardous organic solvents that are used in the preparation of PLGA based sustained release formulations.

Generally, the silk-based drug delivery composition described herein comprises a therapeutic agent encapsulated in a substantially silk matrix, wherein the silk matrix has a cylindrical geometry and the therapeutic agent is present in the lumen of the silk matrix. The therapeutic agent can be in the form of a solid, liquid, or gel.

In some embodiments, the composition is in the form of a silk tube or rod, and the therapeutic agent is present in the lumen of the silk tube or rod. The terms “tube” and “rod” are used interchangeably herein and refer to a cylindrical structure having a lumen therein. Ends of the silk tube can be closed to retain the therapeutic agent within the lumen. It is to be understood that the entire amount of the therapeutic agent needs not be in the lumen of the silk tube. Some of the therapeutic agent can be present, e.g., dispersed or encapsulated, in the walls of the silk tube. Accordingly, in some embodiments, at least 50% (e.g., at least 55%, at least 60%, at least 65%, at least 70%, at least 75%, at least 80%, at least 85%, at least 90%, at least 95%) of the therapeutic agent is within the lumen of the silk tube. In some embodiments, the entire amount of the therapeutic agent is in the lumen of the silk tub, i.e., 100% of the therapeutic agent is in the lumen of the silk tube.

The composition can be used as an implant or as an injectable formulation. Advantageously, silk-based drug delivery compositions herein can be used to administered the therapeutic agent once every 1-6 months (e.g., once every 1-2 months, once every 3-6 months) instead of the usually more frequent administration (e.g., 1-3 times or more a week) of therapeutic agents for treatment of cancer.

In some embodiments, the therapeutic agent can be any agent known in the art for treatment of cancer. In some embodiments, the therapeutic agent can be a therapeutic agent for treatment of breast cancer. In some embodiments, the therapeutic agent can be anastrozole. Anastrozole is a once a day, orally administered tablet. There is no long-term, sustained delivery formulation of anastrozole available.

Provided herein are also kits comprising a silk-based drug delivery composition and instructions for use.

In another aspect, provided herein is a method for treating cancer. The method comprises administering a silk-based drug delivery composition described herein to a subject in need thereof. For administering to a patient, the silk-based drug delivery composition can be formulated with a pharmaceutically acceptable excipient or carrier. The therapeutic agent can be delivered in a therapeutically effective amount over a period of time.

### BRIEF DESCRIPTION OF THE DRAWINGS

FIG. 1 shows SEM cross-sectional morphology of a silk rod (white dashed lines are guides to the eye to highlight expected film boundaries). The inset shows a silk-Anastrozole reservoir rod, rᵢ×rₒ×l=0.75×1.75×20 mm.

FIG. 2 shows an exemplary FSD FTIR spectra collected from film-spun silk tubes and the fit using Gaussian curve shapes and reported peak positions for different molecular conformations of silk fibroin (β: Beta-sheet; RC: Random-coil; βt: β-turns; α: Alphα-helix; SA: Side-chain, aggregate strand).

FIG. 3 shows the aqueous swelling kinetics of film-spun silk tubes 1.5×2.0×20 mm (dᵢ, dₒ, l) in deionized water at room temperature (n=3).

FIG. 4 shows the compiled in vitro anastrozole release rate, R and cumulative release ratio C_{A} for silk reservoir rods with dᵢ, dₒ, l=1.5, 2.0, 20 mm, and effective loading, m_{A}′=1.0 mg/mm (n=3).

FIG. 5 shows the time dependence of rat body mass normalized to the initial body mass (n=3).

FIG. 6 shows the time evolution of in vivo anastrozole plasma concentration in female Sprague-Dawley rats (study groups labeled according to Table 1).

FIG. 7 shows the time evolution of daily in vitro anastrozole release rate (study groups labeled according to Table 1).

FIG. 8 shows the dependence of average anastrozole plasma concentration in female Sprague-Dawley rats to average in vitro daily release rate.

FIG. 9 shows the dependence of effective rod length normalized average in vitro daily release rate on rod dimensions (squares and triangle denote lₑ values of 10 mm and 30 mm, respectively).

FIG. 10 shows in vitro daily anastrozole release rate (squares) and cumulative percent release (diamonds) for silk reservoir rods with dᵢ, dₒ, l=3.17, 3.87, 46 mm, m_{A}′=6.0 mg/mm (n=3). Dashed line shows in vitro target anastrozole release rate.

FIG. 11 shows time evolution of silk fibroin rod dry mass (triangles), apparent β-sheet content measured by FT-IR spectroscopy (diamonds) and apparent mass averaged molecular weight values measured by SEC (squares) as a function of implant duration in rats (n=3, all values normalized to pre-implantation values).

### DETAILED DESCRIPTION OF EXEMPLARY EMBODIMENTS

The present disclosure provides a solution to the problems associated with daily or weekly administration of therapeutic agents for chronic diseases and disorders. The silk-based drug delivery compositions described herein were developed to address the issues associated with repeated injections. In solving this problem, the inventors have demonstrated the use of cylindrical silk-based drug delivery compositions for sustained release of an exemplary breast cancer therapeutic agent, anastrozole, in vitro and in vivo. Anastrozole is a once a day, orally administered tablet. Currently, there is no long-term, sustained delivery formulation of anastrozole available.

Generally, the silk-based drug delivery composition described herein comprises a silk matrix comprising a therapeutic agent, wherein the therapeutic agent is present in a lumen of the silk matrix. Further, the therapeutic agent can be in any form desired. For example, the therapeutic agent can be in the form of a solid, liquid, or gel. In some embodiments, the therapeutic agent is in the form of a solution, powder, a compressed powder or a pellet.

In some embodiments, the silk matrix has a cylindrical geometry. The term “cylindrical” as used herein means having the shape of a cylinder, i.e., a tube with a cross-sectional area and two ends. Accordingly, in some embodiments, the silk-based drug delivery composition is in the form of a silk tube, wherein the therapeutic agent is present in the lumen of the silk tube and the two ends of the silk tube are closed. Generally, the silk tube can be of desired length. For example, length of the silk tube can be from about 1 mm to about 10 cm. In some embodiments, the length of the silk tube can be from about 1 mm to about 40 cm. In some embodiments, the length of the silk tube can be about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm. In some embodiments, length of the silk tube excludes the portion of the silk tube used for closing the ends of the tube. For example, length of the silk tube is length of the lumen therein and excludes the portion of the silk tube that comprises the closed ends.

Without wishing to be bound by a theory, wall thickness of the silk tube can affect the release rate of the therapeutic agent encapsulated in the silk tube. Accordingly, the silk tube can be selected to have a wall thickness that provides a desired rate of release. For example, wall thickness can range from about 50 μm to about 5 mm. In some embodiments, the wall thickness can be from about 50 μm to about 500 μm, from about 50 μm to about 1,000 μm, from about 200 μm to about 300 μm, from about 600 μm to about 800 μm, from about 200 μm to about 800 μm, from about 300 μm to about 700 μm, from about 400 μm to about 600 μm, or about 500 μm. In some embodiments, the wall thickness can be greater than about 1,000 μm. In some embodiments, the wall thickness can be less than about 100 μm. In some embodiments, the wall thickness can be about 0.15 mm, 0.2 mm, 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, or about 4 mm. In some embodiments, the wall thickness can be about 0.09 mm, about 0.10 mm, about 0.15 mm, about 0.21 mm, about 0.24 mm, or about 0.26 mm. Thickness of the silk tube wall can be adjusted by number of layers of silk fibroin present in the wall. For example, silk tube wall can comprise one or more (e.g., one, two, three, four, five, six, seven, eight, nine, ten, eleven, twelve, thirteen, fourteen, fifteen, sixteen, seventeen, eighteen, nineteen, twenty, or more) silk fibroin layers. In some embodiments, the silk tube wall comprises from 1 to 50, 1 to 45, 1 to 40, 1 to 35, 1 to 30, 1 to 25, from 1 to 20, from 1 to 15, or from 1 to 10 silk fibroin layers. In one embodiment, the silk tube wall comprises 9 silk fibroin layers.

Further, thickness of each silk fibroin layer can independently range from about 1 μm to about 1 mm. In some embodiments, thickness of each layer ranges from about 5 μm to about 200 μm, from about 10 μm to about 100 μm, or from 15 to about 50 μm. In some embodiments, thickness of at least one layer is about 20 μm. In some embodiments, thickness of each layer is about 20 μm. In some embodiments, thickness of at least one layer is about 50 μm. In some embodiments, thickness of each layer is about 50 μm.

The overall cross-section of the silk tube can be, for example without limitation, round, substantially round, oval, substantially oval, elliptical, substantially elliptical, triangular, substantially triangular, square, substantially square, hexagonal, substantially hexagonal, or the like. In some embodiments, the overall cross-section of the silk tube is substantially round. The diameter of the overall cross-section of the silk tube can range from about 0.1 mm to about 20 mm. In some embodiments, the diameter of the overall cross-section of the silk tube can range from about 0.5 mm to about 10 mm, from about 1 mm to about 7.5 mm, or from about 1.5 mm to about 5 mm. In some embodiments, the diameter of the overall cross-section of the silk tube is about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, or about 5 mm. In some embodiments, diameter of the silk tube can be about 1.93 mm, about 1.95 mm, about 2.06 mm, about 2.17 mm, about 2.43 mm, or about 2.66 mm. The total diameter of the silk tube is also referred to as dₒ herein.

The silk tube can have a lumen extending therethrough. The lumen can have the same cross-section as the overall cross-section of the silk tube silk or a cross-section that is different than the overall cross-section of the silk tube. For example, the cross-section of the lumen can be round, substantially round, oval, substantially oval, elliptical, substantially elliptical, triangular, substantially triangular, square, substantially square, hexagonal, substantially hexagonal, or the like. In some embodiments, cross-section of the lumen is substantially round.

It is understood that the diameter of the lumen can vary along the length of the lumen. Without limitations, the diameter can be from about 100 nm to about 10 mm. In some embodiments, the diameter can be from about 0.1 mm to about 5 mm, from about 0.5 mm to about 3 mm, from about 0.75 mm to about 2.5 mm, from about 1 mm to about 2 mm. In some embodiments, diameter of the lumen is about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm. about 3 mm, about 3.25 mm, or about 3.5 mm. The diameter of the lumen is also referred to as dᵢ herein.

Generally, the lumen can be about the same length as the length of the silk tube. However, in some embodiments, length of the lumen is shorter than the length of the silk tube because ends of the silk tube are used to close the tube to retain the therapeutic agent in the lumen. Accordingly, the length of the lumen can be from about 1 mm to about 10 cm. In some embodiments, the length of the lumen can be from about 1 mm to about 40 cm. In some embodiments, the length of the lumen can be about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm. Length of the lumen is also referred to as effective length of the silk tube herein.

What is meant by “substantially round” is that the ratio of the lengths of the longest to the shortest perpendicular axes of the cross-section is less than or equal to about 1.5. Substantially round does not require a line of symmetry. In some embodiments, the ratio of lengths between the longest and shortest diameter of the cross-section is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. It is to be understood that the discussion of substantially round applies to both the overall cross-section of the silk tube and the cross-section of the lumen of the silk tube.

In some embodiments, the silk tube can be porous, wherein the silk tube can have a porosity of at least about 30%, at least about 40%, at least about 50%, at least about 60%, at least about 70%, at least about 80%, at least about 90%, or higher. Too high porosity can yield a silk tube with lower mechanical properties, but with faster release of a therapeutic agent. However, too low porosity can decrease the release of a therapeutic agent. One of skill in the art can adjust the porosity accordingly, based on a number of factors such as, but not limited to, desired release rates, molecular size and/or diffusion coefficient of the therapeutic agent, and/or concentrations and/or amounts of silk fibroin in the silk tube. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The porous silk tube can have any pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pores of a silk tube can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. In some embodiments, the silk fibroin can be swollen when the silk fibroin tube is hydrated. The sizes of the pores or the mesh size can then change depending on the water content in the silk fibroin. The pores can be filled with a fluid such as water or air.

Methods for forming pores in a silk matrix are known in the art, e.g., porogen-leaching method, freeze-drying method, and/or gas-forming method. Such methods are described, e.g., in U.S. Pat. App. Nos.: US 2010/0279112, US 2010/0279112, and U.S. Pat. No. 7,842,780, the contents of which are incorporated herein by reference in their entirety.

Though not meant to be bound by a theory, silk tube porosity, structure and mechanical properties can be controlled via different post-spinning processes such as heat treatment, alcohol treatment, air-drying, lyophilization and the like. Additionally, any desirable release rates, profiles or kinetics of the therapeutic agent can be controlled by varying processing parameters, such as film thickness, silk molecular weight, concentration of silk in the silk tube, beta-sheet conformation structures, silk II beta-sheet crystallinity, or porosity and pore sizes.

After preparation, the silk-based drug delivery composition described herein can be sterilized using conventional sterilization process such as radiation based sterilization (i.e. gamma-ray), chemical based sterilization (ethylene oxide), autoclaving, or other appropriate procedures. In some embodiments, sterilization process can be with ethylene oxide at a temperature between from about 52° C. to about 55° C. for a time of 8 or less hours. The silk based drug delivery can also be processed aseptically. Sterile drug delivery composition can packaged in an appropriate sterilize moisture resistant package for shipment.

As used herein, the term “silk fibroin” or “fibroin” includes silkworm silk and insect or spider silk protein. See e.g., Lucas et al., Adv. Protein Chem. 1958, 13, 107-242. Any type of silk fibroin can be used according to aspects of the present invention. There are many different types of silk produced by a wide variety of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Other Silks include transgenic silks, genetically engineered silks (recombinant silk), such as silks from bacteria, yeast, mammalian cells, transgenic animals, or transgenic plants, and variants thereof. See for example, WO 97/08315 and U.S. Pat. No. 5,245,012, content of both of which is incorporated herein by reference in its entirety. In some embodiments, silk fibroin can be derived from other sources such as spiders, other silkworms, bees, synthesized silk-like peptides, and bioengineered variants thereof. In some embodiments, silk fibroin can be extracted from a gland of silkworm or transgenic silkworms. See for example, WO2007/098951, content of which is incorporated herein by reference in its entirety.

In some embodiments, the composition comprises low molecular weight silk fibroin fragments, i.e., the composition comprises a population of silk fibroin fragments having a range of molecular weights, characterized in that: no more than 15% of total weight of the silk fibroin fragments in the population has a molecular weight exceeding 200 kDa, and at least 50% of the total weight of the silk fibroin fragments in the population has a molecular weight within a specified range, wherein the specified range is between about 3.5 kDa and about 120 kDa. Without limitations, the molecular weight can be the peak average molecular weight (Mp), the number average molecular weight (Mn), or the weight average molecular weight (Mw)

As used herein, the phrase “silk fibroin fragments” refers to polypeptides having an amino acid sequence corresponding to fragments derived from silk fibroin protein, or variants thereof. In the context of the present disclosure, silk fibroin fragments generally refer to silk fibroin polypeptides that are smaller than the naturally occurring full length silk fibroin counterpart, such that one or more of the silk fibroin fragments within a population or composition are less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, etc. In some embodiments, “a composition comprising silk fibroin fragments” encompasses a composition comprising non-fragmented (i.e., full-length) silk fibroin polypeptide, in additional to shorter fragments of silk fibroin polypeptides. Silk fibroin fragments described herein can be produced as recombinant proteins, or derived or isolated (e.g., purified) from a native silk fibroin protein or silk cocoons. In some embodiments, the silk fibroin fragments can be derived by degumming silk cocoons under a specified condition selected to produce the silk fibroin fragments having the desired range of molecular weights. Low molecular weight silk fibroin compositions are described in U.S. Provisional Application Ser. No. 61/883,732, filed on Sep. 27, 2013, content of which is incorporated herein by reference in its entirety.

In some embodiments, the silk fibroin is substantially depleted of its native sericin content (e.g., 5% (w/w) or less residual sericin in the final extracted silk). Alternatively, higher concentrations of residual sericin can be left on the silk following extraction or the extraction step can be omitted. In some embodiments, the sericin-depleted silk fibroin has, e.g., about 1% (w/w) residual sericin, about 2% (w/w) residual sericin, about 3% (w/w) residual sericin, about 4% (w/w), or about 5% (w/w) residual sericin. In some embodiments, the sericin-depleted silk fibroin has, e.g., at most 1% (w/w) residual sericin, at most 2% (w/w) residual sericin, at most 3% (w/w) residual sericin, at most 4% (w/w), or at most 5% (w/w) residual sericin. In some other embodiments, the sericin-depleted silk fibroin has, e.g., about 1% (w/w) to about 2% (w/w) residual sericin, about 1% (w/w) to about 3% (w/w) residual sericin, about 1% (w/w) to about 4% (w/w), or about 1% (w/w) to about 5% (w/w) residual sericin. In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected.

Without wishing to be bound by a theory, properties of the silk-based drug delivery compositions disclosed herein can be modify through controlled partial removal of silk sericin or deliberate enrichment of source silk with sericin. This can be accomplished by varying the conditions, such as time, temperature, concentration, and the like for the silk degumming process.

Degummed silk can be prepared by any conventional method known to one skilled in the art. For example, B. mori cocoons are boiled for about up to 90 minutes, generally about 10 to 60 minutes, in an aqueous solution. In one embodiment, the aqueous solution is about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution.

In some embodiments, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 5,718-26, content of all which is incorporated herein by reference in their entirety. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, i.e., solvents other than water.

Generally, any amount of silk fibroin can be present in the solution used for forming the silk tubes or for closing the ends of the silk tube. For example, amount of silk fibroin in the solution can be from about 0.1% (w/v) to about 90% (w/v). In some embodiments, the amount of silk fibroin in the solution can be from about 1% (w/v) to about 75% (w/v), from about 1% (w/v) to about 70% (w/v), from about 1% (w/v) to about 65% (w/v), from about 1% (w/v) to about 60% (w/v), from about 1% (w/v) to about 55% (w/v), from about 1% (w/v) to about 50% (w/v), from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the silk fibroin in the solution is about 25% (w/v). In some embodiments, the silk fibroin in the solution is about 0.5 (w/v) to about 30% (w/v), about 4% (w/v) to about 16% (w/v), about 4% (w/v) to about 14% (w/v), about 4% (w/v) to about 12% (w/v), about 4% (w/v) to about 0% (w/v), about 6% (w/v) to about 8% (w/v). In some embodiments, the silk fibroin solution has a silk fibroin concentration of from about 5% to about 40%, from 10% to about 40%, or from about 15% to about 40% (w/v). In some embodiments, the silk fibroin solution has a silk fibroin concentration of about 5% (w/v), about 7.5% (w/v), about 8% (w/v), about 10% (w/v), about 12.5% (w/v), about 15% (w/v), about 17.5% (w/v), about 20% (w/v), about 22.5% (w/v), about 25% (w/v), about 27.5% (w/v), about 30% (w/v), about 32.5% (w/v), about 35% (w/v), about 37.5% (w/v), about 40% (w/v), about 42.5% (w/v), about 45% (w/v), about 47.5% (w/v), or about 50% (w/v). Exact amount of silk in the silk solution can be determined by drying a known amount of the silk solution and measuring the mass of the residue to calculate the solution concentration.

Generally, any amount of silk fibroin can be present in the silk-based drug delivery composition disclosed herein. For example, amount of silk fibroin in the silk-based drug delivery composition can be from about 1% (w/w) to about 90% (w/w). In some embodiments, the amount of silk fibroin in the composition can be from about 0.1% (w/w) to about 75% (w/w), from about 1% (w/w) to about 70% (w/w), from about 1% (w/w) to about 65% (w/w), from about 1% (w/w) to about 60% (w/w), from about 1% (w/w) to about 55% (w/w), from about 1% (w/w) to about 50% (w/w), from about 1% (w/w) to about 45% (w/w), from about 1% (w/w) to about 40% (w/w), from about 1% (w/w) to about 35% (w/w), from about 1% (w/w) to about 30% (w/w), from about 1% (w/w) to about 25% (w/w), from about 1% (w/w) to about 20% (w/w), from about 1% (w/w) to about 15% (w/w), from about 1% (w/w) to about 10% (w/w), from about 5% (w/w) to about 25% (w/w), from about 5% (w/w) to about 20% (w/w), from about 5% (w/w) to about 15% (w/w). In some embodiments, the silk fibroin in the composition is about 25% (w/w). In some embodiments, the silk in the composition is about 0.5 (w/w) to about 30% (w/w), about 2% (w/w) to about 8% (w/w), about 2% (w/w) to about 7% (w/w), about 2% (w/w) to about 6% (w/w), about 2% (w/w) to about 5% (w/w), about 3% (w/w) to about 4% (w/w).

Without wishing to be bound by a theory, molecular weight of silk or the silk fibroin concentration used for preparing the silk tube can have an effect on properties of the silk tube, such as swelling ratio, degradation, drug release kinetics and the like.

The silk fibroin for making the silk tubes can be modified for different applications or desired mechanical or chemical properties of the silk tube. One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142).

Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk matrix can be combined with a chemical, such as glycerol, that, e.g., affects flexibility and/or solubility of the matrix. See, e.g., WO 2010/042798, Modified Silk films Containing Glycerol.

In some embodiments, the silk fibroin can be modified with positively/negatively charged peptides or polypeptides, such poly-lysine and poly-glutamic acid. While possible, it is not required that every single silk fibroin molecule in the composition be modified with a positively/negatively charged molecule. Methods of derivatizing or modifying silk fibroin with charged molecules are described in, for example, PCT application no. PCT/US2011/027153, filed Mar. 4, 2011, content of which is incorporated herein by reference in its entirety.

Ratio of modified silk fibroin to unmodified silk fibroin can be adjusted to optimize one or more desired properties of the composition, such as drug release rate or kinetics, degradation rate, and the like. Accordingly, in some embodiments, ratio of modified to unmodified silk fibroin in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the composition comprises a molar ratio of modified to unmodified silk fibroin of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, the composition comprises a molar ratio of modified to unmodified silk fibroin of, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, the composition comprises a molar ratio of modified to unmodified silk fibroin of e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

Optionally, the conformation of the silk fibroin in the silk tube can be further altered after formation of the silk tube. Without wishing to be bound by a theory, the induced conformational change alters the crystallinity of the silk fibroin in the tube, e.g., Silk II beta-sheet crystanllinity. This can alter the rate of release of the therapeutic agent from the silk fibroin tube. The conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low content using conditions at 4° C. (a helix dominated silk I structure), to highest content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Biomacromolecules, 2011, 12,1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with sheer stress. The sheer stress can be applied, for example, by passing the silk composition through a needle. Other methods of inducing conformational changes include applying an electric field, applying pressure, or changing the salt concentration.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin in the composition has a protein structure that substantially includes β-turn and β-strand regions. Without wishing to be bound by a theory, the silk 3 sheet content can impact function and in vivo longevity of the composition. It is to be understood that composition including non-β sheet content (e.g., e-gels) can also be utilized. In aspects of these embodiments, the silk fibroin in the composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the composition has a protein structure including, e.g., at least 10% β-turn and β-strand regions, at least 20% β-turn and β-strand regions, at least 30% β-turn and β-strand regions, at least 40% (3-turn and β-strand regions, at least 50% β-turn and β-strand regions, at least 60% β-turn and (3-strand regions, at least 70% β-turn and β-strand regions, at least 80% β-turn and β-strand regions, at least 90% β-turn and β-strand regions, or at least 95% β-turn and β-strand regions. In yet other aspects of these embodiments, the silk fibroin in the composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk 3 sheet content, from less than 10% to ˜55% can be used in the silk-based drug delivery composition.

In some embodiments, the silk fibroin in the composition has a protein structure that is substantially-free of α-helix and random coil regions. In aspects of these embodiments, the silk fibroin in the composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin in the composition has a protein structure that substantially includes β-turn and β-strand regions. In aspects of these embodiments, the silk fibroin in the composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the composition has a protein structure including, e.g., at least 10% β-turn and β-strand regions, at least 20% β-turn and β-strand regions, at least 30% β-turn and β-strand regions, at least 40% β-turn and β-strand regions, at least 50% (3-turn and β-strand regions, at least 60% β-turn and β-strand regions, at least 70% β-turn and (3-strand regions, at least 80% β-turn and β-strand regions, at least 90% β-turn and β-strand regions, or at least 95% β-turn and β-strand regions. In yet other aspects of these embodiments, the silk fibroin in the composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions.

In some embodiments, the silk fibroin in the composition has a protein structure that is substantially-free of α-helix and random coil regions. In aspects of these embodiments, the silk fibroin in the composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin solution can comprise one or more (e.g., one, two, three, four, five or more) additives. Without limitations, presence of one or more additives in the silk fibroin solution used to prepare the drug delivery compositions can alter the release kinetics of the therapeutic agent from the silk-based drug delivery compositions, e.g., silk tubes, described herein. Without wishing to be bound by a theory, presence of additives in the silk-based drug delivery composition can provide a diffusion barrier to regulate the release of the therapeutic agent from the composition. The additive can be covalently or non-covalently linked with silk fibroin in the silk tube and can be integrated homogenously or heterogeneously within the wall of the silk tube. In some embodiments, the additive can be coated on a surface of the silk tube.

An additive can be selected from small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. Total amount of additives in the solution can be from about 0.1 wt % to about 70 wt %, from about 5 wt % to about 60 wt %, from about 10 wt % to about 50 wt %, from about 15 wt % to about 45 wt %, or from about 20 wt % to about 40 wt %, of the total silk fibroin in the solution.

In some embodiments, an additive is a biocompatible polymer. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, alginate, polyaspartic acid, any derivatives thereof and any combinations thereof. Other exemplary biocompatible polymers amenable to use according to the present disclosure include those described for example in U.S. Pat. No. 6,302,848; No. 6,395,734; No. 6,127,143; No. 5,263,992; No. 6,379,690; No. 5,015,476; No. 4,806,355; No. 6,372,244; No. 6,310,188; No. 5,093,489; U.S. Pat. No. 387,413; No. 6,325,810; No. 6,337,198; No. U.S. Pat. No. 6,267,776; No. 5,576,881; No. 6,245,537; No. 5,902,800; and No. 5,270,419, content of all of which is incorporated herein by reference.

In some embodiments, the biocompatible polymer is PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to Ser. No. 10/000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multiarmed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the biocompatible polymer is a peptide, oligopeptide or a protein. In some embodiments, the biocompatible polymer is albumin. Albumin is a simple protein found in serum and has a molecular weight of about 66,000 Daltons. Albumin is produced in the liver and is the most abundant blood plasma protein. Albumin polypeptides are important in regulating blood volume by maintaining appropriate colloid osmotic pressure. Human serum albumin is a monomer of 585 amino acid residues, and includes three homologous a-helical domains: domain I, domain II and domain III. Each domain contains 10 helices and is divided into antiparallel six-helix and four-helix subdomains. Deletion studies suggest that domain III alone is sufficient for binding to FcRn (Chaudhury et al., Biochemistry 2006, 45:4983-4990). A truncated human albumin that does not bind FcRn and has a low serum level has been identified (Andersen et al., Clin Biochem., 2010, 43(45):367-72. Epub 2009 Dec. 16).

Albumin is known to bind and carry a wide variety of small molecules, including lipid soluble hormones, bile salts, unconjugated bilirubin, fatty acids, calcium, ions, transferrin, hemin, and tryptophan. Albumin also binds various drugs such as Warfarin, phenobutazone, clofibrate and phenytoin, and its binding can alter the drugs' pharrnacokinetic properties.

The albumin can be a naturally occurring albumin, an albumin related protein or a variant thereof such as a natural or engineered variant. Variants include polymorphisms, fragments such as domains and subdomains, fragments and/or fusion proteins. An albumin can comprise the sequence of an albumin protein obtained from any source. Typically the source is mammalian such as human or bovine. In some embodiments, the n one the serum albumin is human serum albumin (“HSA”). The term “human serum albumin” includes a serum albumin having an amino acid sequence naturally occurring in humans, and variants thereof. The HSA coding sequence is obtainable by known methods for isolating cDNA corresponding to human genes, and is also disclosed in, for example, EP 0 073 646 and EP 0 286 424, content of both of which is incorporated by reference in their entirety. A fragment or variant can be functional or non-functional. For example, a fragment or variant can retain the ability to bind to an albumin receptor such as FcRn to at least 10, 20, 30, 40, 50, 60, 70, 80, 90 or 100% of the ability of the parent albumin (from which the fragment or variant derives) to bind to the receptor. Relative binding ability can be determined by methods known in the art such as surface plasmon resonance studies.

The albumin can be a naturally-occurring polymorphic variant of human albumin or of a human albumin analogue. Generally, variants or fragments of human albumin will have at least 5%, 10%, 15%, 20%, 30%, 40%, 50%, 60%, 70%, (preferably at least 80%, 90%, 95%, 100%, 105% or more) of human albumin's ligand binding activity (for example FcRN-binding), mole for mole.

The albumin can comprise the sequence of bovine serum albumin. The term “bovine serum albumin” includes a serum albumin having an amino acid sequence naturally occurring in cows, for example as taken from Swissprot accession number P02769, and variants thereof as defined herein. The term “bovine serum albumin” also includes fragments of full-length bovine serum albumin or variants thereof, as defined herein.

A number of proteins are known to exist within the albumin family. Accordingly, the albumin can comprise the sequence of an albumin derived from one of serum albumin from African clawed frog (e.g., see Swissprot accession number P08759-1), bovine (e.g., see Swissprot accession number P02769-1), cat (e.g., see Swissprot accession number P49064-1), chicken (e.g., see Swissprot accession number P19121-1), chicken ovalbumin (e.g., see Swissprot accession number P01012-1), cobra ALB (e.g., see Swissprot accession number Q91134-1), dog (e.g., see Swissprot accession number P49822-1), donkey (e.g., see Swissprot accession number QSXLE4-1), European water frog (e.g., see Swissprot accession number Q9YGH6-1), blood fluke (e.g., see Swissprot accession number AAL08579 and Q95VB7-1), Mongolian gerbil (e.g., see Swissprot accession number O35090-1 and JC5838), goat (e.g., see Swissprot accession number B3VHM9-1 and as available from Sigma as product no. A2514 or A4164), guinea pig (e.g., see Swissprot accession number Q6WDN9-1), hamster (see DeMarco et al. (2007). International Journal for Parasitology 37(11): 1201-1208), horse (e.g., see Swissprot accession number P35747-1), human (e.g., see Swissprot accession number P02768-1), Australian Lung-fish (e.g., see Swissprot accession number P83517), macaque (Rhesus monkey) (e.g., see Swissprot accession number Q28522-1), mouse (e.g., see Swissprot accession number P07724-1), North American bull frog (e.g., see Swissprot accession number P21847-1), pig (e.g., see Swissprot accession number P08835-1), pigeon (e.g. as defined by Khan et al, 2002,1112. J. Biol. Macromol, 30(3-4), 171-8), rabbit (e.g., see Swissprot accession number P490 65-1), rat (e.g., see Swissprot accession number P02770-1), salamander (e.g., see Swissprot accession number Q8UW05-1), salmon ALB1 (e.g., see Swissprot accession number P21848-1), salmon ALB2 (e.g., see Swissprot accession number Q03156-1), sea lamprey (e.g., see Swissprot accession number Q91274-1 and O42279-1) sheep (e.g., see Swissprot accession number P14639-1), Sumatran orangutan (e.g., see Swissprot accession number Q5NVH5-1), tuatara (e.g., see Swissprot accession number Q8JIA9-1), turkey ovalbumin (e.g., see Swissprot accession number O73860-1), Western clawed frog (e.g., see Swissprot accession number Q6D.I95-1), and includes variants and fragments thereof as defined herein.

Many naturally occurring mutant forms of albumin are known. Many are described in Peters, (1996, All About Albumin: Biochemistry, Genetics and Medical Applications, Academic Press, Inc., San Diego, Calif., p. 170-181), content of which is incorporated herein by reference. A variant as defined herein can be one of these naturally occurring mutants such as those described in Minchiotti et al., Hum Mutat 2008, 29(8): 1007-16, content of which is incorporated herein by reference in its entirety.

A “variant albumin” refers to an albumin protein wherein at one or more positions there have been amino acid insertions, deletions, or substitutions, either conservative or non-conservative, provided that such changes result in an albumin protein for which at least one basic property, for example binding activity (type of and specific activity e.g. binding to bilirubin or a fatty acid such as a long-chain fatty acids, for exampleoleic (C18:1), palmitic (C16:0), linoleic (C18:2), stearic (C18:0), arachidonic (C20:4) and/or palmitoleic (C16:1)), osmolarity (oncotic pressure, colloid osmotic pressure), behaviour in a certain pH-range (pH-stability) has not significantly been changed. “Significantly” in this context means that one skilled in the art would say that the properties of the variant can still be different but would not be unobvious over the ones of the original protein, e.g. the protein from which the variant is derived. Such characteristics can be used as additional selection criteria in the invention.

The term albumin also encompasses albumin variants, such as genetically engineered forms, mutated forms, and fragments etc. having one or more binding sites that are analogous to a binding site unique for one or more albumins as defined above. By analogous binding sites in the context of the invention are contemplated structures that are able to compete with each other for binding to one and the same ligand structure.

In some embodiments, the albumin can be human serum albumin extracted from serum or plasma, or recombinant human albumin (rHA) produced by transforming or transfecting an organism with a nucleotide coding sequence encoding the amino acid sequence of human serum albumin, including rHA produced using transgenic animals or plants. In one embodiment, albumin is bovine serum albumin, includes variants and fragments thereof.

Other additives suitable for use with the present disclosure include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard, Cell Mol Life Sci. 2003, 60(1):119-32; Hersel U. et al. Biomaterials 2003, 24(24):4385-415); biologically active ligands; and substances that enhance or exclude particular varieties of cellular or tissue ingrowth. Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β1 and the like.

In some embodiments, the silk fibroin solution for making the film-spun silk tube or coating the ends comprises one or more therapeutic agents. The therapeutic agent in the silk fibroin solution can be same or different from that is present in the lumen of the silk tube.

Generally, any therapeutic agent can be encapsulated in the silk based drug delivery compositions described herein. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), or mixtures or combinations thereof, including, for example, DNA nanoplexes, siRNA, shRNA, aptamers, ribozymes, decoy nucleic acids, antisense nucleic acids, RNA activators, and the like.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritic antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As noted above, any therapeutic agent can be encapsulated. In some embodiments, the therapeutic agent(s) for use in the present disclosure include, but are not limited to, those requiring relatively frequent dosing. For example, those used in the treatment of diabetes.

In some embodiments, the therapeutic agent is an agent known in the art for treatment of cancer.

In some embodiments, the therapeutic agent is an agent known in the art for treatment of breast cancer. Exemplary therapeutic agents known in the art for treatment of breast cancer include, but are not limited to, adrenal corticosteroid inhibitors, such as aminoglutethimide (Cytadren); alkylating agents, such as cyclophosphamide (Cytoxan, Cytoxan lyophilized, Neosar), thiotepa (Thioplex); androgens and anabolic steroids such as fluoxymesterone (Androxy and Halotestin); antibiotics/antineoplastics, such as doxorubicin (Adriamycin); antimetabolites, such as fluorouracil (Adrucil), capecitabine (Xeloda), and gemcitabine (Gemzar); aromatase inhibitors, such as anastrozole (Arimidex), exemestane (Aromasin), and letrozole (Femara); EGFR inhibitors and HER2 inhibitors, such as lapatinib (Tykerb); estrogen receptor antagonists, such as fulvestran (Faslodex); estrogens, such as esterified estrogens (Estratab and Menest); HER2 inhibitors, such as trastuzumab (Herceptin) and pertuzumab (Perjeta); immunosuppressants, such as methotrexate (Trexall); mitotic inhibitors, such as paclitaxel (Onxol and Taxol), protein-bound paclitaxel (Abraxane), docetaxel (Docefrez, Taxotere), ixabepilone (Ixempra), vinblastine (Velban), and eribulin (Halayen); mTOR inhibitors or selective immunosuppressants, such as everolimus (Afinitor); selective estrogen receptor modulators, such as tamoxifen (Nolvadex, Soltamox) and toremifene (Fareston); and VEGF/VEGFR inhibitors, such as bevacizumab (Avastin).

Additional exemplary agents for treatment of breast cancer include, for example, those described in U.S. Pat. App. Pub. No. 20030013145; No. 20030087265; No. 20040029114; No. 20060246415; and No. 20070065845; and U.S. Pat. No. 4,383,985; No. 4,651,749; No. 4,707,438; No. 5,236,844; No. 5,855,889; No. 5,914,238; No. 6,037,129; No. 6,056,690; No. 6,179,786; No. 6,218,131; No. 6,235,486; No. 6,342,483; No. 6,368,796; No. 6,432,707; No. 6,518,237; No. 6,649,342; No. 6,730,477; No. 6,855,554; No. 6,936,424; No. 7,056,663; No. 7,056,674; No. 7,302,292; No. 7,335,467; No. 7,569,345; No. 7,725,170; No. 7,828,732; No. 7,863,001; No. 7,863,011; No. 7,879,614; No. 8,034,565; No. 8,133,737; and No. 8,206,919, content of all of which is incorporated herein by reference in their entirety.

In some embodiments, therapeutic agent is an aromatase inhibitor.

In some embodiments, therapeutic agent is anastrozole.

Generally, any amount of the therapeutic agent can be loaded into the silk matrix to provide a desired amount release over a period of time. For example, from about 0.1 ng to about 1000 mg of the therapeutic agent can be loaded in the silk matrix. In some embodiment, amount of therapeutic agent in the composition is selected from the range about from 0.001% (w/w) up to 95% (w/w), preferably, from about 5% (w/w) to about 75% (w/w), and most preferably from about 10% (w/w) to about 60% (w/w) of the total composition. In some embodiments, amount of amount of the therapeutic agent in the composition is from about 0.01% to about 95% (w/v), from about 0.1% to about 90% (w/w), from about 1% to about 85% (w/w), from about 5% to about 75% (w/w), from about 10% to about 65% (w/w), or from about 10% to about 50% (w/w), of the total composition.

In some embodiments, amount of the therapeutic agent in the composition is from about 1% to about 99% (w/w), from about 0.05% to about 99% (w/w), from about 0.1% to about 90% (w/w), from about 0.5% to about 85% (w/w), from about 5% to about 80% (w/w), from about 10% to about 60% (w/w) of the total composition. In some embodiments, amount of the therapeutic agent in the composition is from about 0.1% to about 99% (w/w), from about 1% to about 90% (w/w), from about 2% to about 80% (w/w), from about 5% to about 75% (w/w), from about 5% to about 50% (w/w), from about 0.055% to about 0.1% (w/w) of the total composition.

In some embodiments, amount of the therapeutic agent in the silk tube is from about 0.5 mg/mm to about 2.5 mg/mm, from about 0.75 mg/mm to about 2 mg/mm, or from about 0.8 mg/mm to about 1.5 mg/mm of silk tube or lumen length. In some embodiments, amount of the therapeutic agent in the silk tube is about 0.5 mg/mm, about 0.6 mg/mm, about 0.7 mg/mm, about 0.8 mg/mm, about 0.9 mg/mm, about 1 mg/mm, about 1.1 mg/mm, about 1.2 mg/mm, about 1.3 mg/mm, about 1.4 mg/mm, or about 1.5 mg/mm of silk tube or lumen length.

The inventors have discovered inter alia that the therapeutic agent is released in a sustained release manner from the silk-based drug delivery compositions described herein. In other words, the silk-based drug delivery composition described herein is a sustained delivery composition. As used herein, the term “sustained delivery” refers to continual delivery of a therapeutic agent in vivo or in vitro over a period of time following administration. For example, sustained release can occur over a period of at least about 3 days, at least about a week, at least about two weeks, at least about three weeks, at least about four weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months or longer. In some embodiments, the sustained release can occur over a period of more than one month or longer. In some embodiments, the sustained release can occur over a period of at least about three months or longer. In some embodiments, the sustained release can occur over a period of at least about six months or longer. In some embodiments, the sustained release can occur over a period of at least about nine months or longer. In some embodiments, the sustained release can occur over a period of at least about twelve months or longer. Sustained delivery of the therapeutic agent in vivo can be demonstrated by, for example, the continued therapeutic effect of the agent over time. Alternatively, sustained delivery of the therapeutic agent can be demonstrated by detecting the presence or level of the therapeutic agent or a metabolite thereof in vivo over time. By way of example only, sustained delivery of the therapeutic agent, upon administration, can be detected by measuring the amount of therapeutic agent or a metabolite thereof present in blood serum, a tissue or an organ of a subject.

The release rate of a therapeutic agent from the silk-based drug delivery composition can be adjusted by a number of factors such as silk tube composition and/or concentration of silk fibroin used in making the silk tube, porous property of the silk tube, molecular size of the therapeutic agent, and/or interaction of the therapeutic agent with the silk in the silk tube. For example, if the therapeutic agent has a higher affinity with the silk matrix, the release rate is usually slower than the one with a lower affinity with the silk matrix. Additionally, when a silk matrix has larger pores, the encapsulated therapeutic agent is generally released from the silk matrix faster than from a silk matrix with smaller pores.

The release profiles of the therapeutic agent from the silk-based drug delivery composition can be modulated by a number of factors such as amounts and/or molecular size of the therapeutic agents loaded in the silk tube, porosity of the silk tube, amounts of silk fibroin in the silk tube and/or contents of beta-sheet conformation structures in the silk tube, binding affinity of the therapeutic agent to the silk tube, and any combinations thereof.

The silk-based drug delivery composition can provide or release an amount of the therapeutic agent, which provides a therapeutic effect similar to as provided by a recommended dosage of the therapeutic agent for the same period of time. For example, if the recommended dosage for the therapeutic agent is once daily, then the silk-based drug delivery composition releases that amount of therapeutic agent, which is sufficient to provide a similar therapeutic effect as provided by the once daily dosage.

Without limitations, daily release of the therapeutic agent can range from about 1 ng/day to about 1000 mg/day. For example, amount released can be in a range with a lower limit of from 1 to 1000 (e.g., every integer from 1 to 1000) and upper limit of from 1 to 1000 (e.g. every integer from 1 to 1000), wherein the lower and upper limit units can be selected independently from ng/day, μg/day, mg/day, or any combinations thereof.

In some embodiments, daily release can be from about 1 μg/day to about 10 mg/day, from about 10 μg/day to about 5 mg/day, from about 100 μg/day to about 2.5 mg/day, from about 250 μg/day to about 1 mg/day, or from about 250 μg/day to about 750 μg/day. In some embodiments, daily release of the therapeutic agent is from about 500 μg/day to about 700 μg/day. In some embodiments, daily release of the therapeutic agent is about 600 μg/day. In some embodiments, daily release of the therapeutic agent is from about 150 μg/da to about 225 μg/day. In some embodiments, daily release can be from about 600 μg/day to about 1000 μg/day. In one embodiment, daily release can be about 965 μg/day. In one embodiment, daily release of the therapeutic agent is about 190 μg/day.

The silk-based drug delivery compositions disclosed herein release about the same amount of the therapeutic agent every day for a period of time. For example, daily release of the therapeutic agent be within 25% (e.g., within 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%) of the average daily release over a period of time.

The inventors have discovered that release of the therapeutic agent from the silk reservoir implant or silk injectable reservoir composition follows near zero-order release kinetics over a period of time. For example, near zero-order release kinetics can be achieved over a period of one week, two weeks, three weeks, four weeks, one month, two months, three months, four months, five months, six months, twelve months, one year or longer.

In some embodiments, no significant apparent initial burst release is observed from the drug delivery composition described herein. Accordingly, in some embodiments, the initial burst of the therapeutic agent within the first 48, 24, 18, 12, or 6 hours of administration is less than 25%, less than 20%, less than 15%, less than 10%, less than 9%, less than 8%, less than 7%, less than 6%, less than 5%, less than 4%, less than 3%, less than 2%, or less than 1% of the total amount of therapeutic agent loaded in the drug delivery composition. In some embodiments, there is no initial burst of therapeutic agent within the first 6 or 12 hours, 1, 2, 3, 4, 5, 6, 7 days, 1 and 2 weeks of administration.

The silk-based drug delivery compositions disclosed herein retain their overall structural integrity after administration, e.g., implantation, to a subject and provide zero-order sustained delivery for a period of time. However, the silk based drug delivery compositions can completely biodegrade over longer durations with favorable biodegradation profile for controlled, sustained delivery applications.

The silk-based drug delivery composition can stabilize the activity, e.g., bioactivity, of a therapeutic agent under a certain condition, e.g., under an in vivo physiological condition. See, for example, U.S. Provisional Application No. 61/477,737, filed Apr. 21, 2011 and International Patent Application No. PCT/US2012/034643, filed Apr. 23, 2012, the content of both of which is incorporated herein by reference in its entirety. Accordingly, the silk-based drug delivery composition can increase the in vivo half-life of the therapeutic agent. For example, in vivo half-life of an encapsulated therapeutic agent can increase by at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or more relative to the non-encapsulated therapeutic agent. In some embodiments, in vivo half-life of the encapsulated therapeutic agent is at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or longer than the in vivo half-life of the therapeutic agent when not encapsulated in the silk matrix.

Without wishing to be bound by theory, the silk-based drug delivery composition can provide a longer therapeutic effect. Stated another way, an increase in in vivo half-life of a therapeutic agent can allow loading of a smaller amount of the therapeutic agent for the same duration of therapeutic effect. Accordingly, encapsulating a therapeutic agent in a silk matrix can increase the duration of effect for the therapeutic agent. For example, amount of therapeutic agent encapsulated in the silk-based drug delivery composition provides a therapeutic effect for a period of time, which is longer than when the same amount of therapeutic agent is administered without the silk-based drug delivery composition. In some embodiments, duration of therapeutic effect is at least one day, at least two days, at least three days, at least four days, at least five days, at least six days, at least seven days, at least one week, at least two weeks, at least three weeks, at least four weeks, at least one month, at least two months, at least three months, at least four months, at least five months, at least six months, at least seven months, at least eight months, at least nine months, at least ten months, at least eleven months, at least twelve months, at least thirteen month, at least fourteen months, at least fifteen months, at least sixteen months, at least seventeen months, at least eighteen months, at least nineteen months, at least twenty months, at least twenty one months, at least twenty two months, at least twenty three months, at least twenty four months, or longer than the duration of effect when the therapeutic agent is administered without the silk-based drug delivery composition.

In some embodiments, the duration of therapeutic effect from a single dosage is at least one day, at least two days, at least three days, at least four days, at least five days, at least six days, at least seven days, at least one week, at least two weeks, at least three weeks, at least four weeks, at least one month, at least two months, at least three months, at least four months, at least five months, at least six months, at least seven months, at least eight months, at least nine months, at least ten months, at least eleven months, at least twelve months, at least thirteen month, at least fourteen months, at least fifteen months, at least sixteen months, at least seventeen months, at least eighteen months, at least nineteen months, at least twenty months, at least twenty one months, at least twenty two months, at least twenty three months, at least twenty four months, or longer.

Accordingly, the silk-based drug delivery compositions described herein can comprise the therapeutic agent in an amount which is less than the amount recommended for one dosage of the therapeutic agent. For example, if the recommended dosage of the therapeutic agent is X amount then the silk matrix can comprise a therapeutic agent in an amount of about 0.9×, about 0.8×, about 0.7×, about 0.6×, about 0.5×, about 0.4×, about 0.3×, about 0.2×, about 0.1× or less. Without wishing to be bound by a theory, this can allow administering a lower dosage of the therapeutic agent in a silk matrix to obtain a therapeutic effect which is similar to when a higher dosage is administered without the silk matrix.

In some embodiments, amount of the therapeutic agent dispersed or encapsulated in the silk matrix can be more than the amount generally recommended for one dosage of the same therapeutic agent administered for a particular indication. For example, if the recommended dosage of the therapeutic agent is X amount then the silk matrix can encapsulate a therapeutic agent in an amount of about 1.25×, about 1.5×, about 1.75×, about 2×, about 2.5×, about 3×, about 4×, about 5×, about 6×, about 7×, about 8×, about 9×, about 10×, about 20×, about 30×, about 40×, about 50×, about 60×, about 70×, about 80×, about 90×, about 100×, about 200×, about 300×, about 400×, about 500×, about 600×, about 700× or more. Without wishing to be bound by a theory, this can allow administering the therapeutic agent in a silk matrix to obtain a therapeutic effect which is similar to one obtained with multiple administration of the therapeutic agent administered without the silk matrix described herein.

In some embodiments, the amount of the therapeutic agent encapsulated in the silk matrix can be essentially the same amount recommended for one dosage of the therapeutic agent. For example, if the recommended dosage of the therapeutic agent is X amount, then the silk-based composition can comprise about X amount of the therapeutic agent. Since the silk-based drug delivery compositions described herein can increase the duration of effect for the therapeutic agent, this can allow less frequent administration of the therapeutic agent to obtain a therapeutic effect over a longer period of time.

Furthermore, the silk-based drug delivery composition can increase bioavailability of the encapsulated therapeutic agent. As used herein, the term “bioavailability” refers to the amount of a substance available at a given site of physiological activity after administration. Bioavailability of a given substance is affected by a number of factors including but not limited to degradation and absorption of that substance. Administered substances are subject to excretion prior to complete absorption, thereby decreasing bioavailability. In some embodiments, bioavailability of an encapsulated therapeutic agent can increase by at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or more relative to the non-encapsulated therapeutic agent.

Without wishing to be bound by a theory, silk-based drug delivery compositions can allow the frequency of administration of the therapeutic agent to be reduced by a factor of F=(Y2−Y1)/Y2, wherein Y1 is the duration of the therapeutic effect produced by the current dosage of the therapeutic agent without silk matrix recommended for a particular indication, and Y2 is the duration of the therapeutic effect produced by the same amount of the therapeutic agent present in a silk-based drug delivery composition described herein. Frequency of administration for the silk matrix encapsulated therapeutic agent can be calculated using the formula:

Frequency of administration=Z×F [1]

wherein Z is number of administrations of the therapeutic agent in the absence of the silk matrix over a given period of time.

For example, if the duration of the therapeutic effect produced by the current dosage of the therapeutic agent without silk matrix recommended for a particular indication is one month (Y1=1 month) and the duration of the therapeutic effect produced by the same amount of the therapeutic agent present in a silk-based drug delivery composition described herein is two month, then the frequency of administration is reduced by a factor of ½ (e.g., Y2=2 months, and Y1=1 month). The frequency of administration is reduced to about once every two months. That is, instead of having an administration of the therapeutic agent once a month with the current administration protocol, the methods and/or compositions of the invention can reduce frequency of administration to about once every two months. Similarly, if the frequency of administration is reduced by a factor of ⅔ (e.g., Y2=3 months, and Y1=1 month), the methods and/or compositions described herein can reduce frequency of administration to about once every 3 months.

In some embodiments, the frequency of administration of the therapeutic agent can be reduced by a factor of at least about 1/700, at least about 1/600, at least about 1/500, at least about 1/250, at least about 1/225, at least about 1/200, at least about 1/175, at least about 1/150, at least about 1/125, at least about 1/100, at least about 1/90. at least about 1/80, at least about 1/70, at least about 1/60, at least about 1/50, at least about 1/30, at least about 1/25, at least about 1/20, at least about 1/19, at least about 1/18, at least about 1/17, at least about 1/16, at least about 1/15, at least about 1/14, at least about 1/13, at least about 1/12, at least about 1/11, at least about 1/10, at least about 1/9, at least about ⅛, at least about 1/7, at least about ⅙, at least about ⅕, at least about ¼, at least about ⅓, at least about ½, at least about 1/1.75, at least about 1/1.5, at least about 1/1.25, at least about 1/1.1, or more.

Generally, silk tubes can be made using any method known in the art. For example, tubes can be made using molding, dipping, electrospinning, gel spinning, and the like. Gel spinning involves winding an aqueous solution of silk around a reciprocating rotating mandrel. Final gel-spun silk tube porosity, structure and mechanical properties could be controlled via different post-spinning processes such as alcohol (e.g., methanol, ethanol, etc. . . . ) treatment, air-drying or lyophilization.

Gel spinning is described in Lovett et al. (Biomaterials 2008, 29(35):4650-4657) and the construction of gel-spun silk tubes is described in PCT application no. PCT/US2009/039870, filed Apr. 8, 2009, content of both of which is incorporated herein by reference in their entirety. Construction of silk tubes using the dip-coating method is described in PCT application no. PCT/US2008/072742, filed Aug. 11, 2008, content of which is incorporated herein by reference in its entirety.

An exemplary method for preparing silk tubes is a method previously described by the inventors in U.S. Provisional Application No. 61/613,185, filed Mar. 20, 2012, and PCT application No. PCT/US2013/030206, filed Mar. 11, 2013, contents of which are incorporated herein by reference in their entirety. The method described in U.S. Ser. No. 61/613,185 and PCT/US2013/030206 is based on a novel and nonobvious modification of the gel spinning technique as described in PCT application no. PCT/US2009/039870. The film-spun silk tube preparation method described in U.S. Ser. No. 61/613,185 and PCT/US2013/03020 is different from that described in PCT/US2009/039870. Mainly, heating the silk during spinning unexpectedly provides a silk tube with a controlled morphology. Accordingly, the tube preparation technique described in U.S. Ser. No. 61/613,185 and PCT/US2013/03020 is termed “film spinning,” as it involves a heat treatment step using an in-line heating element to transition the silk spinning solution into a tubular film with controlled morphology and a more controlled tube wall thickness for applications involving controlled delivery of therapeutic agents.

Generally, the film spinning method for forming a silk tube comprises: (i) delivering a silk fibroin solution onto a mandrel which is reciprocated horizontally while being rotated along its longitudinal axis to form a silk coating thereon and heating the silk coating while the mandrel is rotating to form a silk film on the rotating mandrel. The mandrel can have an elongated structure with a longitudinal axis. The inventors have discovered that simultaneous rotation of the mandrel and treatment of film with heat unexpectedly results in coating thickness uniformity.

Without limitations, the delivering and heating steps can be repeated one or more times (e.g., 1, 2, 3, 4, 5, 6, 7, 8, 9, 10 or more) to form one or more coatings of the silk film. In some embodiments, the delivering and heating steps are repeated at least 5, at least 10, at least 50, at least 100, at least 250, at least 500, at least 1000, at least 5000, at least 10000 or more times. In some embodiments, the delivering and heating steps are repeated until a desired wall thickness for the film-spun silk tube is obtained.

The mandrel can be made of any material known to one of skill in the art. For example, mandrel can be a stainless steel mandrel coated with a synthetic fluoropolymer.

The mandrel can have a rotational speed of about 0 to about 1000 rpm and an axial movement speed of about 0 to about 1000 mm/s.

The silk fibroin solution can be delivered onto the mandrel using any method known in the art. For example, the silk fibroin solution can be applied using an applicator. In some embodiments, the applicator can be a syringe containing the supply of the silk solution.

The silk fibroin solution can be delivered onto the mandrel using a needle. A needle of any gauge can be used for delivery. For example, the needle can be of at least 21 Gauge. In some embodiment, needle is of gauge from about 18 to about 30.

Without limitations, the silk fibroin solution can be delivered onto the mandrel at any flow rate. For example, a 30 wt % silk solution can be delivered at a flow rate of 0.03 mL/min to dispense about 2 μL of silk solution per millimeter of axial displacement of a 2.7 mm diameter wire rotating at a speed of 70 rpm.

The silk coating can be heated simultaneously while the silk fibroin solution is being delivered onto the mandrel or after delivery has finished. For example, the silk coating can be treated with heat within 5 seconds, within 10 second, within 14 second, within 25 seconds, within 30 seconds, within 35 second, within 40 seconds, within 45 seconds, within 50 seconds, within 55 seconds, within 1 minute, within 2 minutes, within 3 minutes, within 4 minutes, within 5 minutes, within 6 minutes, within 7 minutes, within 8 minutes, within 9 minutes, within 10 minutes, within 15 minutes, within 20 minutes, within 25 minutes, within 30 minutes, within 45 minutes, or within 1 hour of delivery of the silk solution onto the mandrel.

Any temperature higher than room temperature can be used for heat treating the silk film on the support structure. For example, temperature for the heat treatment can range from about 30° C. to about 90° C. In some embodiments, temperature for the heat treatment can range from about 35° C. to about 80° C., from about 40° C. to about 75° C., from about 50° C. to about 70° C., or from about 55° C. to about 65° C. In some embodiments, temperature for the heat treatment is 67±3° C., or 47±3° C.

Further, the silk film on the support structure can be heat treated any period of time. For example, heat treatment can be for a period of about 1 minute to about 6 hours. In some embodiments, heat treatment can be for from about 10 minutes to about 300 minutes. In some embodiments, heat treatment can be for about 1, 2, 3, 4, 5, 10, 20, 30, or 60 minutes.

For loading into the silk tubes, a therapeutic agent can be in any form suitable for the particular method to be used for loading. For example, the therapeutic agent can be in the form of a solid, liquid, or gel. In some embodiments, the therapeutic agent is in the form of a solution, powder, a compressed powder or a pellet.

In some embodiments, the silk tube can be optionally hydrated before loading with the therapeutic agent. For example, the silk tube can be incubated in deionized water until completely hydrated. In some embodiments, the silk tube can be incubated in deionized water for 5, 10, 15, 20, 30, 45, 60, 90, 120, 150, 180, 210, 240, 270, 300 minutes or more. The tube can be hydrated at room temperature or at higher temperatures. Accordingly, in some embodiments, the tube can be hydrated at a temperature from about 15° C. to about 80° C. In some embodiments, the tube can be hydrated at a temperature about 60° C. Without wishing to be bound by a theory, hydrating the silk tube before loading can swell or soften the tube thus promoting loading.

In some embodiments, the silk tube can be open at both ends during loading. In this case, the hydrated silk tube can be held horizontally using tweezers, while the therapeutic agent is loaded from one end in solution, powder or pellet format using an appropriately sized pipetter, spatula or tweezers, respectively. In some embodiments, one end of the tube can be clamped before loading of the therapeutic agent using for example, pinch valves, clips or wrenches. The tube clamped on one end can be held vertically, while the therapeutic agent is loaded from the open end in solution, powder or pellet format using an appropriately sized pipetter, spatula or tweezers, respectively. Following loading, the open end(s) of the tube can be clamped using for example, pinch valves, clips or wrenches.

Following loading of therapeutic agent, clamped, hydrated silk tubes can be dried at a suitable temperature (e.g., 20° C. or higher temperatures) in ambient conditions for a suitable duration (e.g. 30 min or longer) to allow complete drying of the tube and the loaded therapeutic agent. Alternatively, clamped, hydrated silk tubes can be dried under accelerated drying conditions (e.g. in vacuum, or under gas flow for a suitable duration to allow complete drying of the tube and the loaded drug (e.g. for 10 min or longer). Drying conditions can be selected to maximize stability of the therapeutic agent.

After drying, the closed ends of the silk tube can be coated with a silk fibroin solution, e.g., via dip coating to obtain silk reservoir implants or silk injectable reservoirs. Dip coating can be repeated several times until the desired coating thickness is achieved. Without wishing to be bound by a theory, coating the closed ends helps in forming a tight seal and prevents dose dumping. The tube ends can be coated with a silk fibroin solution using any method known in the art. For example, the silk fibroin solution can be sprayed on the closed ends or the closed ends dipped into the silk fibroin solution. In one embodiment, closed ends of the tube are dipped into a silk fibroin.

All aforementioned steps to produce silk tube loaded with the therapeutic agent can be performed under aseptic conditions. For example, the film spinning, methanol treatment, hydration, drug loading, heat treatment and dip coating procedures can be conducted aseptically inside a laminar flow hood.

In one embodiment, loading of the therapeutic agent into silk tubes comprises: (i) optionally hydrating the silk tube; (ii) loading the therapeutic agent into the tube and tube end clamping; (iii) drying the silk tube; and (iv) dip coating of tube ends.

In yet another aspect, provided herein is a method for sustained delivery in vivo of a therapeutic agent. The method comprising administering a silk-based drug delivery composition described herein to a subject. Without wishing to be bound by a theory, the therapeutic agent can be released in a therapeutically effective amount daily.

As used herein, the term “therapeutically effective amount” means an amount of the therapeutic agent which is effective to provide a desired outcome. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Furthermore, therapeutically effective amounts will vary, as recognized by those skilled in the art, depending on the specific disease treated, the route of administration, the excipient selected, and the possibility of combination therapy. In some embodiments, the therapeutically effective amount can be in a range between the ED50 and LD50 (a dose of a therapeutic agent at which about 50% of subjects taking it are killed). In some embodiments, the therapeutically effective amount can be in a range between the ED50 (a dose of a therapeutic agent at which a therapeutic effect is detected in at least about 50% of subjects taking it) and the TD50 (a dose at which toxicity occurs at about 50% of the cases). In some embodiments, the therapeutically effective amount can be an amount determined based on the current dosage regimen of the same therapeutic agent administered in a non-silk matrix. For example, an upper limit of the therapeutically effective amount can be determined by a concentration or an amount of the therapeutic agent delivered or released on the day of administration with the current dosage of the therapeutic agent in a non-silk matrix; while the lower limit of the therapeutically effective amount can be determined by a concentration or an amount of the therapeutic agent on the day at which a fresh dosage of the therapeutic agent in a non-silk matrix is required. Guidance regarding the efficacy and dosage which will deliver a therapeutically effective amount of a compound can be obtained from animal models of condition to be treated.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, and immunological assays.

The dosage can be determined by a physician and adjusted, as necessary, to suit observed effects of the treatment. Generally, the therapeutic agents are administered so that the therapeutic agent is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For protein therapeutic agents, one preferred dosage is 0.1 mg/kg of body weight (generally 10 mg/kg to 20 mg/kg).

As disclosed herein, the silk-based drug delivery can provide a therapeutically effective amount of the therapeutic agent to a subject for a period of time which is similar to or longer than the period of time when the therapeutic agent is administered without the silk-based drug delivery composition. For example, amount of therapeutic agent released over a day provides a similar therapeutic effect as provided by the recommended daily dosage of the therapeutic agent when administered without the silk-based drug delivery composition.

For administration to a subject, the silk-based drug delivery composition can be formulated in pharmaceutically acceptable compositions which comprise a drug delivery composition, formulated together with one or more pharmaceutically acceptable carriers (additives) and/or diluents. The drug delivery composition can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery composition. See, for example, Urquhart, et al., Ann. Rev. Pharmacol. Toxicol. 1984, 24: 199-236; Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

As used herein, the term “administered” refers to the placement of a drug delivery composition into a subject by a method or route which results in at least partial localization of the pharmaceutically active agent at a desired site. A drug delivery composition described herein can be administered by any appropriate route which results in effective treatment in the subject, i.e., administration results in delivery to a desired location in the subject where at least a portion of the pharmaceutically active agent is delivered. Exemplary modes of administration include, but are not limited to, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments, a drug delivery composition described herein can be implanted in a subject. As used herein, the term “implanted,” and grammatically related terms, refers to the positioning of the silk-based drug delivery composition in a particular locus in the subject, either temporarily, semi-permanently, or permanently. The term does not require a permanent fixation of the silk-based drug delivery composition in a particular position or location. Exemplary in vivo loci include, but are not limited to site of a wound, trauma or disease.

In some embodiments, the silk-based drug delivery compositions described herein are suitable for in vivo delivery to a subject by an injectable route. One delivery route is injectable, which includes intravenous, intramuscular, subcutaneous, intraperitoneal, intrathecal, epidural, intra-arterial, intra-articular and the like. Other delivery routes, such as topical, oral, rectal, nasal, pulmonary, vaginal, buccal, sublingual, transdermal, transmucosal, otic or intraocular, could also be practiced.

For injection, silk-based drug delivery compositions can be aspirated into a syringe and injected through a needle of gauge of about 10 to about 34 or about 12 to about 30. An exemplary delivery route is injection with a fine needle, which includes subcutaneous, ocular and the like. By fine needle is meant needles of at least 10 Gauge size, typically between about 12 Gauge and about 30 Gauge and above. In some embodiments, the fine needles can be at least as fine as 10 Gauge, 12 Gauge, 14 Gauge, 16 Gauge, 18 Gauge, 19 Gauge, 21 Gauge, at least as fine as 22 Gauge, at least as fine as 23 Gauge, at least as fine as 24 Gauge, at least as fine as 25 Gauge, at least as fine as 26 Gauge, or at least as fine as 28 Gauge.

Without limitations, method of sustained delivery described herein can be used for administering, to a subject, a pharmaceutical agent that requires relatively frequent administration. For example, a pharmaceutically active agent that requires administration at least once every three months, at least once every two months, at least once every week, at least once daily for a period of time, for example over a period of at least one week, at least two weeks, at least three weeks, at least four weeks, at least one month, at least two months, at least three months, at least four months, at least five months, at least six months, at least one years, at least two years or longer.

As is known in the art, many therapeutic agents for treatment of chronic disorders or conditions require relatively frequent dosing. Thus, provided herein is method for treatment of a chronic disease or disorder in subject. The method comprises administering a silk-based drug delivery composition described herein or a pharmaceutical composition comprising silk-based drug delivery composition described herein to subject in need thereof. The silk-based drug delivery comprises a therapeutic agent that requires frequent administration for treatment of chronic disease or condition under consideration.

Exemplary chronic diseases include, but are not limited to, autoimmune disease including autoimmune vasculitis, cartilage damage, CIDP, Cystic Fibrosis, diabetes (e.g., insulin diabetes), graft vs. host disease, Hemophilia, infection or other disease processes, inflammatory arthritis, inflammatory bowel disease, inflammatory conditions resulting from strain, inflammatory joint disease, Lupus, lupus, Multiple Sclerosis, Myasthenia Gravis, Myositis, orthopedic surgery, osteoarthritis, Parkinson's Disease, psioriatic arthritis, rheumatoid arthritis, Sickle Cell Anemia, sprain, transplant rejection, trauma, and the like.

By “treatment, prevention or amelioration” is meant delaying or preventing the onset of such a disorder or reversing, alleviating, ameliorating, inhibiting, slowing down or stopping the progression, aggravation or deterioration the progression or severity of such a condition. In some embodiments, at least one symptom is alleviated by at least 20%, at least 30%, at least 40%, at least 50%, at least 60%, at least 70%, at least 80%, at least 90%, or at least 95% but not 100%, i.e. not a complete alleviation. In some embodiments, at least one symptom is completely alleviated.

In some embodiments, subject is need of treatment for cancer. As used herein, the term “cancer” or “tumor” refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). The cancer can be an early stage cancer without local or systemic invasion or the cancer can be an invasive cancer and/or a cancer capable of metastasis. Typical cancers are solid or hematopoietic cancers such as breast, stomach, oesophageal, sarcoma, ovarian, endometrium, bladder, cervix uteri, rectum, colon, lung or ORL cancers, paediatric tumours (neuroblastoma, glioblastoma multiforme), lymphoma, leukaemia, myeloma, seminoma, Hodgkin and malignant hemopathies. In some embodiments, the cancer is selected from the group consisting of leukemia, lymphoma, melanoma, lung cancer, bowel cancer, colon cancer, rectal cancer, colorectal cancer, brain cancer, liver cancer, pancreatic cancer, breast cancer, prostate cancer, testicular cancer and retinoblastoma. In some preferred embodiment, the cancer is a solid cancer, preferably a breast cancer or a prostate cancer, more preferably a breast cancer.

As used herein, the term “treatment of cancer” refers to any act intended to extend life span of patients such as therapy and retardation of the disease. The treatment can be designed to eradicate the tumor, to stop the progression of the tumor, to prevent the occurrence of metastasis, to promote the regression of the tumor and/or to prevent muscle invasion of cancer. Preferably, the term “treatment of cancer” as used herein, refers to the prevention or delay of metastasis formation, disease progression and/or systemic invasion.

In some embodiments, the method further comprises selecting a subject for treatment of cancer, i.e., a subject having or suspected of developing a cancer.

Exemplary embodiments of the invention can be also described by any one of the following numbered paragraphs.
1. A sustained delivery composition, the composition comprising
   (i) a silk matrix comprising a lumen; and
   (ii) an anti-cancer agent;
   wherein the anti-cancer agent is in the lumen; and two ends of the lumen are closed to retain the anti-cancer agent within the lumen.
2. The composition of paragraph 1, wherein the silk matrix is a cylindrical shape.
3. The composition of paragraph 1 or 2, wherein the silk matrix has a length of from about 1 mm to about 10 cm.
4. The composition of any of paragraphs 1-4, wherein the silk matrix is has a length of about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm.
5. The composition of any of paragraphs 1-5, wherein the silk matrix has a wall thickness of from about 50 μm to about 5 mm.
6. The composition of any of paragraphs 1-6, wherein the silk matrix has a wall thickness of about 0.09 mm, about 0.10 mm, about 0.15 mm, about 0.21 mm, about 0.24 mm, about 0.25 mm, about 0.26 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, or about 4 mm.
7. The composition of any of paragraphs 1-6, wherein the silk matrix has a diameter from about from about 0.5 mm to about 10 mm.
8. The composition of any of paragraphs 1-7, wherein the silk matrix has a diameter of about 1 mm, about 1.25 mm, about 1.5 mm, about 1.75 mm, about 1.93 mm, about 1.95 mm, about 2 mm, about 2.06 mm, about 2.17 mm, about 2.25 mm, about 2.43 mm, about 2.5 mm, about 2.66 mm, about 2.75 mm, about 3 mm, about 3.25 mm, about 3.5 mm, about 3.75 mm, about 4 mm, about 4.25 mm, about 4.5 mm, about 4.75 mm, or about 5 mm.
9. The composition of any of paragraphs 1-8, wherein the lumen has a diameter from about from about 100 nm to about 10 mm.
10. The composition of any of paragraphs 1-9, wherein the lumen has a diameter of about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm. about 3 mm, about 3.25 mm, or about 3.5 mm.
11. The composition of any of paragraphs 1-10, wherein the lumen has a length of from about 1 mm to about 10 cm.
12. The composition of any of paragraphs 1-11, wherein the lumen has a length of about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm.
13. The composition of any of paragraphs 1-12, wherein silk fibroin in the silk matrix comprises silk II beta-sheet crystallinity of at least 5%.
14. The composition of any of paragraphs 1-13, wherein silk fibroin in the silk matrix comprises silk II beta-sheet crystallinity of about 47%.
15. The composition of any of paragraphs 1-14, wherein the anti-cancer agent is an anti-breast cancer agent.
16. The composition of any of paragraphs 1-15, wherein the anti-cancer agent is selected from the group consisting of adrenal corticosteroid inhibitors, alkylating agents, androgens and anabolic steroids, antibiotics/antineoplastics, antimetabolites, aromatase inhibitors, EGFR inhibitors and HER2 inhibitors, estrogen receptor antagonists, estrogens, HER2 inhibitors, immunosuppressants, mitotic inhibitors, mTOR inhibitors, selective immunosuppressants, selective estrogen receptor modulators, and VEGF/VEGFR inhibitors, and any combinations thereof
17. The composition of any of paragraphs 1-16, wherein the anti-cancer agent is anastrozole.
18. The composition of any of paragraphs 1-17, wherein the composition comprises from about 0.01% to about 95%(w/w) of the anti-cancer agent.
19. The composition of any of paragraphs 1-18, wherein the composition comprises from about 0.5 mg to about 2.5 mg of the anti-cancer agent per mm of length of the silk matrix or the lumen.
20. The composition of any of paragraphs 1-19, wherein the composition comprises about 0.5 mg, about 0.6 mg, about 0.7 mg, about 0.8 mg, about 0.9 mg, about 1 mg, about 1.1 mg, about 1.2 mg, about 1.3 mg, about 1.4 mg, or about 1.5 mg of the anti-cancer agent per mm of length of the silk matrix or the lumen.
21. The composition of any of paragraphs 1-20, wherein the silk matrix further comprises a biocompatible polymer.
22. The composition of any of paragraphs 1-21, wherein the composition is implantable or injectable.
23. The composition of any of paragraphs 1-22, wherein the silk matrix has the dimensions:
   (i) a length of about 10 mm, a lumen of diameter about 1.5 mm, and an outer diameter of about 2.0 mm;
   (ii) a length of about 20 mm, a lumen diameter of about 1.5 mm, and an outer diameter about 2.0 mm;
   (iii) a length of about 20 mm, a lumen diameter of about 1.0 mm, and an outer diameter about 2.0 mm;
   (iv) a length of about 20 mm, a lumen diameter of about 1.5 mm, and an outer diameter about 3.5 mm;
   (v) a length of about 46 mm, a lumen diameter of about 3.2 mm, and an outer diameter about 3.9 mm; or
   (vi) a length of about 36 mm, a lumen diameter of about 3.9 mm, and an outer diameter about 3.5 mm.
24. The composition of any of paragraphs 1-23, wherein the composition comprises:
   (i) the silk matrix having a length of about 10 mm, a lumen of diameter about 1.5 mm, and an outer diameter of about 2.0 mm; and about 1.3 mg or about 1.4 mg of the anti-cancer agent per mm of length of the silk matrix;
   (ii) the silk matrix having a length of about 20 mm, a lumen diameter of about 1.5 mm, and an outer diameter about 2.0 mm; and about 0.6 mg of the anti-cancer agent per mm of length of the silk matrix;
   (iii) the silk matrix having a length of about 20 mm, a lumen diameter of about 1.0 mm, and an outer diameter about 2.0 mm; and about 0.8 mg or about 0.7 mg of the anti-cancer agent per mm of length of the silk matrix;
   (iv) the silk matrix having a length of about 20 mm, a lumen diameter of about 1.5 mm, and an outer diameter about 3.5 mm; about 0.9 mg or about 1.3 mg of the anti-cancer agent per mm of length of the silk matrix; or
   (v) the silk matrix having a length of about 46 mm, a lumen diameter of about 3.2 mm, and an outer diameter about 3.9 mm; and about 6 mg of the anti-cancer agent per mm of length of the silk matrix.
25. The composition of any of paragraphs 1-22, wherein the silk matrix has the dimensions:
   (i) a lumen length of about 10 mm, a lumen diameter of about 1.75 mm, and an outer diameter of about 1.93 mm;
   (ii) a lumen length of about 20 mm, a lumen diameter of about 1.75 mm, and an outer diameter of about 1.95 mm;
   (iii) a lumen length of about 30 mm, a lumen diameter of about 1.76 mm, and an outer diameter of about 2.06 mm, and wall thickness of about 0.15 mm;
   (iv) a lumen length of about 40 mm, a lumen diameter of about 1.75 mm, and an outer diameter of about 2.17 mm;
   (v) a lumen length of about 40 mm, a lumen diameter of about 1.95 mm, and an outer diameter of about 2.43 mm;
   (vi) a lumen length of about 40 mm, a lumen diameter of about 2.14 mm, and an outer diameter of about 2.66 mm;
   (vii) a lumen length of about 46 mm, a lumen diameter of about 3.2 mm, and an outer diameter of about 3.9 mm; or
   (viii) a lumen length of about 36 mm, a lumen diameter of about 3.5 mm, and an outer diameter of about 3.9 mm.
26. The composition of any of paragraphs 1-25, wherein the composition provides sustain release of the anti-cancer agent over a period of at least about a week.
27. The composition of any of paragraphs 1-26, wherein anti-cancer agent is released from the composition at a rate of from about 1 μg/day to about 10 mg/day.
28. The composition of paragraph 27, wherein the anti-cancer agent is released from the silk matrix at a rate of about 600 to about 1000 μg/day.
29. The composition of any of paragraphs 1-28, wherein the anti-cancer agent has duration of therapeutic effect which is at least one day longer relative to duration of therapeutic effect in the absence of the silk matrix.
30. A pharmaceutical composition comprising a sustained delivery composition of any of paragraphs 1-29 and a pharmaceutically acceptable carrier.
31. A method for treating cancer in a subject, the method comprising administering to a subject in need thereof a composition of any of paragraphs 1-29.
32. The method of paragraph 31, wherein administration frequency of the composition is less than when the same amount of the anti-cancer agent is administered in the absence of the silk matrix.
33. The method of paragraph 32, wherein the administration frequency is reduced by a factor of ½ relative to when the anti-cancer agent is administered in the absence of the silk matrix.
34. The method of any of paragraphs 31-33, wherein said administration is no more than once a month, no more than once every two week, no more than once every three weeks, no more than once a month, no more than once every two months, no more than once every four months or no more once every six months.
35. A drug delivery device comprising the composition of any of paragraphs 1-29.
36. The drug delivery device of paragraph 35, wherein the drug delivery device is a syringe with an injection needle.
37. The drug delivery device of paragraph 36, wherein the device is an implant.
38. A kit comprising a composition of any of paragraphs 1-28, or a drug delivery device of any of paragraphs of 35-37.
39. The kit of paragraph 38, further comprising at least a syringe and an injection needle.
40. The kit of any of paragraphs 38-39, further comprising an anesthetic.
41. The kit of any of paragraphs 38-40, further comprising an antiseptic agent.
42. The kit of any of paragraphs 38-41, further comprising instruction for use.
43. A method of preparing a sustained delivery composition of any of paragraphs 1-29, the method comprising:
   (i) forming a silk tube, wherein forming the silk tube comprises:
      a. delivering, with an applicator, a silk solution onto a support structure, wherein the support structure is an elongated structure with a longitudinal axis, and wherein the support structure is reciprocated horizontally while being rotated along its longitudinal axis to form a silk coating thereon;
      b. heating the silk coating, while rotating the wire, to form a silk film; and
      c. optionally repeating the delivering and heating steps to form one or more coatings of silk film thereon;
   (ii) inducing a conformational change in the silk coating;
   (iii) optionally hydrating the silk tube;
   (iv) loading the silk tube with an anti-cancer agent;
   (v) closing ends of the silk tube such that the therapeutic agent is sealed therein.

### SOME SELECTED DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the terms “essentially” and “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “essentially” means a proportion of at least about 90%, at least about 95%, at least about 98%, at least about 99% or more, or any integer between 90% and 100%. In some embodiments, the term “essentially” can include 100%.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated may be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

A number of silk protein-based material formats (e.g., micro/nanoparticulate suspensions, injectable hydrogels, aerogels, implants (Rockwood et al., Nature Protocols, 2011, 6, 1612; Wang et al., Biomaterials, 2010, 31, 1025; Yucel et al., Biophysical Journal, 2009, 97, 2044) have been investigated for sustained drug delivery (Wang et al., Biomaterials, 2010, 31, 1025; Guziewicz, Biomaterials, 2011, 32, 2642; Pritchard et al., Expert Opinion on Drug Delivery, 2011, 8, 797) due to their desirable aqueous, ambient temperature processing, high biocompatibility and controllable biodegradation kinetics through molecular structure. For example, 14-day sustained release of model drugs from silk micro/nanosphere suspensions was demonstrated in vitro (Wang et al., Biomaterials, 2010, 31, 1025). Importantly, drug release kinetics from silk spheres depended strongly on drug physicochemical properties, e.g., hydrophobicity, charge, and molecular weight, and the strength or lifetime of silk-drug interactions. In another example, 14-day, near zero-order sustained release of a small molecule drug from silk fibroin-coated pellet implants was demonstrated in vitro (Pritchard et al., Journal of Controlled Release, 2010, 144, 159). Overall, a strict control over the overall dimensions and structure of the formulation and development of minimally invasive administration procedures are essential in demonstrating proof of concept for a silk reservoir, sustained delivery technology.

Silk tubes were previously fabricated mainly for tissue engineering applications, such as complex composite biomaterial matrices, blood vessel grafts and nerve guides, using molding, dipping, electrospinning, and gel spinning (Lovett et al., Biomaterials, 2008, 29, 4650). Gel spinning involves winding an aqueous solution of silk around a reciprocating rotating mandrel. Final gel-spun silk tube porosity and mechanical properties could be manipulated via different post-spinning processes such as methanol treatment, air-drying or lyophilization. A method for the preparation of silk reservoir rods for drug delivery was recently described (Kaplan et al. U.S. Provisional Application No. 61/613,185, 2012). This method involves a combination of film-spinning, which is a modification of the gel-spinning method, drug loading, and dip coating to seal drug loaded tube ends. In film-spinning, the silk spinning solution is injected onto a rotating mandrel at a controlled flow rate, and immediately exposed to a critical heat-treatment step using an in-line heating element to obtain a tubular silk film with uniform and controlled thickness and overall silk II, β-sheet crystallinity. Such tight control over overall dimensions and structure is essential for controlled drug delivery applications. Secondly, hydrated silk film tubes are loaded with a desired drug in powder or solution form followed by clamping of tube ends. Lastly, silk film tube ends are dip coated to ensure a complete seal and prevent dose dumping.

### Materials and Methods

Degummed silk fibers were purchased from Suho Biomaterials Technology (Suzhou, China). Anastrozole, chlorpheniramine and all other chemicals were purchased from Sigma-Aldrich (St. Louis, Mo.).

Regenerated Silk Fibroin Solution:

A 20 wt. % solution of degummed silk fibers in 9.3 M aqueous LiBr was dialyzed against deionized water (ρ≈18.2 MΩ·cm) for 48 hours using Slide-A-Lyzer dialysis cassettes (3 kDa MWCO, Fisher Scientific, Pittsburgh, Pa.). The conductivity of the dialysis water was probed to ensure completion of desalting. The final concentration of the regenerated silk solution was 7±1 wt. %. Silk solution resistivity, pH and high shear viscosity (5 wt. % silk) values were 25±5 kΩ cm, 8.5±0.5 pHU and 3.1±0.5 cP at 25° C., respectively (mean±SD, n=3). The molecular weight distribution was characterized via size exclusion chromatography. One microgram of silk protein was injected into an analytical column (SEC-3, 4.6 mm×300 mm, 300 Å, Agilent, Santa Clara, Calif.) using an Agilent 1200 Series HPLC pump and 1×PBS with 0.05 wt. % NaN₃ as the mobile phase. The molecular weight standards were cytidine (243 Da), bovine serum albumin (67 kDa), γ-globulin (158 kDa) and thyroglobulin (660 kDa). Calculated weight averaged molecular weight (M_{W}) and polydispersity values of the monomer distribution were 198±15 kDa and 19.9±1.1, respectively (mean±SD, n=3). Silk fibroin solution was concentrated to 28-35 wt. % via dialysis against 15-20 wt. % aqueous PEG (10 kDa) for 16-24 hours using 3 kDa MWCO Slide-A-Lyzer dialysis cassettes. Silk concentration was measured gravimetrically and via Bradford Assay to within +0.5 wt. %.

Silk-Anastrozole Reservoir Rods:

A custom set-up was developed for film spinning silk tubes (Kaplan et al., U.S. Provisional Application No. 61/613,185, 2012). Briefly, concentrated silk solution (28-35 wt. %) was injected at a flow rate between 0.15 and 0.50 mm³/s through a narrow gauge needle (≧21 G) onto a PTFE-coated stainless steel wire (McMaster-Carr, Atlanta, Ga.). The injection rate was controlled using a syringe pump (KD Scientific, Holliston, Mass.). During injection, the wire was concomitantly reciprocated horizontally at 0.33 mm/s, while being rotated along its axis at 1 Hz. The motion of the wire was controlled through an AC gear motor (McMaster-Carr, Atlanta, Ga.) connected to another syringe pump (KD Scientific, Holliston, Mass.). Immediately after injection of silk solution, the rotating wire was transferred into a tube oven to heat-treat the silk solution, typically at 80±5° C. for 300 s to obtain a 0.05-0.10 mm thick film. Simultaneous rotation of the wire during the film heat treatment ensured thicknesses uniformity (≦10% thickness variation along tube length). Subsequent coating and drying was repeated until the desired tube diameter was achieved. Silk film tubes were soaked in methanol:water (9:1, v/v) for 60 s/coat to induce silk II, β-sheet crystallinity. Film-spun tubes were swollen in deionized water, removed from the PTFE wire and cut to a desired length (typically 10-40 mm). Anastrozole was loaded in powder form and compacted using a PTFE wire, and ≈5 mm from both ends of the tube were clamped using pinch valves. After drying, each clamped tube end was dip-coated in 28-35 wt. % silk solution at 2 mm/s and dried at 60° C. for 30 min to obtain silk-anastrozole reservoir rods. The whole procedure was conducted aseptically in a biosafety cabinet using non-pyrogenic consumables.

Fourier Transform Infrared Spectroscopy:

Silk secondary structure in film-spun tubes were probed using a Fourier Transform Infrared Spectrometer (Alpha-Eco FT-IR, Bruker, Billerica, Mass.) with a zinc selenide Attenuated Total Reflection (ATR) sampling module. Hydrated film spun tubes were cut along their length and dried under compression at room temperature under vacuum into flat films. Each sample spectrum was the Fourier transform of 128 scans with a resolution of 4 cm⁻¹. For semi-quantitation of the amide I region (1590-1710 cm⁻¹), Fourier Self-Deconvoluted (FSD) spectra was curve fit using OPUS software (Bruker, Billerica, Mass.) according to previously published protocols (Hu et al., Macromolecules, 2008, 41, 3939). The wavenumber assignment to common silk secondary structure forms was: 1610-1635 and 1696-1705 to β-sheet, 1640-1650 to random coil, 1650-1660 to α-helix and 1661-1695 to β-t-urns) The relative contribution of each secondary structure form to the overall molecular conformation was estimated from the ratio of the corresponding peak area to that of the whole FSD spectra.

In Vitro Release Assays:

Silk-anastrozole rods were incubated in 200-400 ml of deionized water containing 0.02 wt. % NaN₃ at 37° C. for the desired duration of the study. The release volume was determined to ensure perfect sink conditions in case of a complete burst release according to:

$$V \geq 10 \frac{L}{s} \qquad [ 2 ]$$

where V, L and S are the release volume, drug loading and aqueous solubility, respectively. At pre-determined time points, 1 ml of the release medium was sampled. After each sampling, the whole medium was exchanged with fresh buffer. No significant degradation of Anastrozole was detected via LC-MS/MS under the studied release conditions during the longest inter-sampling duration of 1 week.

A steady state target release rate of anastrozole, R_{T} was calculated assuming a one-compartment, continuous infusion model:

$${R}_{T} = \frac{ln ( 2 )}{{t}_{1 / 2}} \cdot V \cdot {C}_{ss} \qquad [ 3 ]$$

where t_{1/2} is the terminal elimination half-life, V is the volume of distribution and Cₛₛ is the steady state plasma concentration. A target anastrozole release rate was calculated as 0.6 mg/d for t_{1/2}, V and Cₛₛ values of 50 h, 74 l and 25 ng/ml respectively (AstraZeneca Canada, Inc., ARIMIDEX™, Product Monograph, 2011). Here, Cₛₛ value factors in a reported three-fold anastrozole accumulation.

In Vivo Pharmacokinetics:

A 91-day pharmacokinetic study was conducted on female Sprague-Dawley rats (≧250 g). Test article animals were dosed by a single rod implantation, while positive drug control animals were dosed by a single injection of an ethanol:water (3:7, v/v) solution of equivalent dose. Prior to dosing, all animals were anesthetized by intraperitoneal injection of a cocktail containing ketamine HCl (75 mg/kg) and Xylazine HCl (5 mg/kg). Following anesthesia, the dorsal surface of all animals were shaved and prepared for aseptic administration by wiping with betadine and 70% isopropyl alcohol (3 times each). Animals were placed on a sterile surgical field and covered with Steridrape™. Dosing sites were closed with wound clips. The administration site was circled with indelible ink for future identification of location. Following dosing, all animals were placed in a warmed recovery area and observed until recovered from anesthesia and ambulatory. All animals were observed throughout dosing and each scheduled collection. Body weights were collected weekly in addition to the pre-dose body weight during the duration of the study. Serial blood samples were collected via tail vein or jugular vein at pre-dose, 2 h, 6 h, 24 h (1 d), 2 d, 4 d, 7 d, 10 d, 14 d, 21 d, 28 d, 35 d, 42 d, 49 d, 56 d, 63 d, 70 d, 77 d, 84 d and 91 d and analyzed via LC-MS/MS. Blood samples were stored on wet ice until processed to plasma by centrifugation within 30 min of collection. Plasma were stored at −80° C. until LC-MS/MS analysis.

Bio-Analysis Using Liquid Chromatography-Tandem Mass Spectroscopy:

For bio-analysis, a modification of a previously published protocol on human plasma pharmacokinetics of anastrozole (Mendes et al., Journal of Chromatography B, 2007, 850, 553) was used. Briefly, 150 μl of frozen in vivo sample or blank plasma (for double blank, blank and standards) was completely thawed and briefly centrifuged (2000 G, 3 min, 4° C.). Eight concentration standards between 0.21 to 450 ng/ml were prepared by diluting aqueous Anastrozole solutions 20-fold in blank plasma in microcentrifuge tubes. For extraction, 1 ml diethyl ether:dichloromethane (7:3, v/v) was added to 25 μl of plasma (blank, standard or sample) and 10 μl of aqueous internal standard (100 ng/ml Chlorpheniramine) or deionized water (for double blank) in a glass centrifuge tube using glass serological pipettes and vortexed for 40 s. After a brief centrifugation (2000 G, 2 min, 4° C.), the organic supernatant was transferred into a clean glass centrifuge tube and allowed to dry completely at 40° C. under nitrogen gas flow. The pellet was re-suspended in 200 μl of deionized water, vortexed for 1 min and briefly centrifuged (2000 G, 2 min, 4° C.). One hundred seventy-five microliters of the supernatant was transferred into 96-well plates, capped and placed in the auto-sampler for LC-MS/MS analysis.

The auto-sampler of the LC system was kept at 5° C. Ten microliters of sample was injected into a C₁₈ analytical column (Zorbax Eclipse Plus, 2.1 mm×100 mm, 3.5 μm, Agilent, Santa Clara, Calif.) at 25° C. using an isocratic mobile phase of acetonitrile:methanol:water: acetone (60:20:15:5, v/v/v/v) containing 0.1% of acetic acid and 10 mM of ammonium acetate. The flow rate was 0.4 ml/min. Under these conditions, typical standard retention times were 0.75 min for anastrozole and 0.69 min for chlorpheniramine. Tandem mass spectrometry was performed using an Agilent 6410 triple stage quadrupole mass spectrometer in positive electrospray ionization mode. The spectrometer was operated the in Multiple Reaction Monitoring (MRM) mode using the peak areas of 294.2>225.2 and 275.2>230.1 transitions to quantify anastrozole and chlorpheniramine concentrations, respectively. The source block temperature was set at 300° C. using nitrogen as the collision gas. The MRM parameters were optimized for both anastrozole and chlorpheniramine using the Agilent Optimizer software. An 8-point standard curve was generated for the concentration standard peak areas using a linear least-squares regression with a weighting index of 1/x². Intra-batch accuracy was within 100±20%. The limit of quantitation (LOQ) was 0.6 ng/ml.

Swelling Kinetics:

Time evolution of aqueous swelling ratio of silk tubes (S) was calculated from:

$$q ( t ) = \frac{{m}_{H} ( t )}{{m}_{D}} \qquad [ 4 ]$$

where, m_{H}(t) is the hydrated mass at time t, and nip is the dry mass, respectively.

Partition Coefficient:

Silk tubes (i.d., o.d., l.=1.5, 2.0, 10 mm) were incubated in 0.1 or 1 mg/ml aqueous anastrozole solution at room temperature with mild (˜1 Hz) orbital shaking at a solution (V_{S}) to hydrated silk tube (V_{T}) volumetric ratio, V_{S}/V_{T} 9.0.1 or 1 mg/ml aqueous anastrozole solution containing no silk tubes was run as a control to account for possible anastrozole binding to the container.

Aliquots were collected from the supernatant until apparent equilibration and analyzed for anastrozole concentration via LC-MS/MS. Partition coefficient (K_{d}) was calculated using:

$${K}_{d} = \frac{{V}_{S}}{{V}_{T}} ( \frac{{C}_{B} - {C}_{T}}{{C}_{T}} ) \qquad [ 5 ]$$

where V_{S}, V_{T}, C_{T} and C_{B} are the solution and tube volumes, and apparent equilibrium supernatant concentrations from silk tube positive and silk tube blank samples, respectively.

### Results and Discussion

Reservoir Rod Morphology and Structure:

Silk-anastrozole reservoir rods were prepared using previously described film spinning-end sealing method (Kaplan et al. U.S. Provisional Application No. 61/613,185, 2012) as described in the Experimental section. For the present study, the tube inner diameter, dᵢ was varied between 1.0 and 1.5 mm, while the tube outer diameter, dₒ values were between 2.0 and 3.5 mm, leading to a wall thickness, Δr values between 0.25 to 1.0 mm (typical silk film thickness variation was ≦10%). The rod length, l was 20 mm for all groups except for one in vitro group with 1=40 mm.

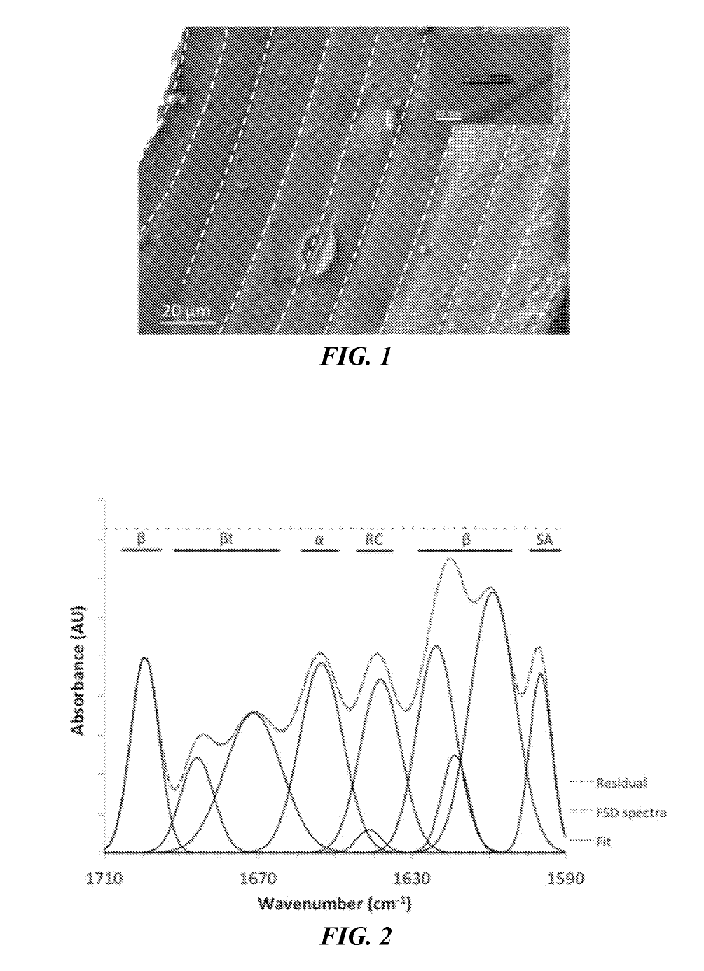

Cross-sectional SEM images of film-spun silk tubes showed uniform layers of silk film coating with no apparent film defects (e.g. micro-cracks) or any evidence of delamination of film layers (FIG. 1) as previously reported in silk films used for controlled delivery (Pritchard et al., Journal of Controlled Release, 2010, 144, 159). The molecular conformation of film-spun silk tubes was investigated via Fourier Transform Infrared (FT-IR) Spectroscopy (FIG. 2). Fourier self-deconvolution of the FT-IR spectra followed by curve fitting according to common secondary structure form peaks identified for the silk protein indicated a high silk II, β-sheet contribution to the overall molecular conformation (≈47%). This value was close to the high β-sheet content measured from silk films treated in methanol:water (9:1, v/v) for 24 h. The relative contribution of β-sheet, β-turn, α-helix and random-coil structure to the overall conformation was 47%, 19%, 14%, 13%, respectively, while 7% of the spectra was attributed to side-chains and aggregated strands.

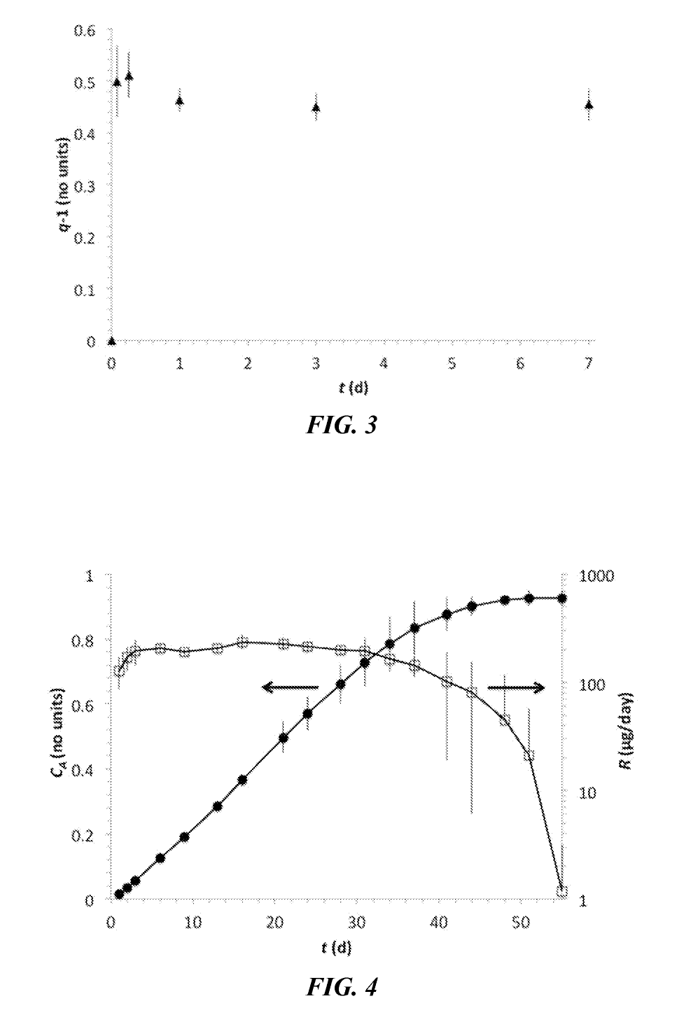

FIG. 3 shows swelling kinetics of 1.5×2.0×20 mm (dᵢ, dₒ, l) film-spun silk tubes in deionized water at room temperature. The aqueous swelling ratio reached a value of q=1.50±0.07 (n=3) within 2 h of incubation, while an insignificantly slight decrease to 1.46±0.02 (n=3) was observed after day 1 and the q value essentially remained constant for the rest of the 1-week incubation. Overall, the film swelling to an apparent equilibrium value of ≈1.5 was essentially immediate in relation to the time frame relevant for the PK studies (e.g., months).

Pharmacokinetics and Biocompatibility:

FIG. 4 shows the time evolution of daily anastrozole release rate, R and cumulative release ratio, C_{A}(t) in a pilot in vitro dissolution test on silk reservoir rods. Here, C_{A}(t)=m_{R}(t)/m_{A}, where m_{R}(t) is the cumulative anastrozole mass released at time t and m_{A} is the total anastrozole load (C_{A}(t)=1 indicates complete release). The overall rod dimensions were 1.5×2.0×20 mm (dᵢ, dₒ, l) with an effective anastrozole load, =m_{A}/lₑ value of 1.0 mg/mm (n=3). Here, lₑ is the effective rod length that excludes the length used for tube end sealing via dip coating. Zero-order release kinetics was observed between days 2 and 37, with an essentially constant R value of 190±31 μg/day (mean±standard deviation) up to a cumulative release value of ≈0.8. Combined with the swelling data, PK results indicate silk film swelling and formation of an equilibrium, linear concentration gradient along the silk film thickness within the first 2 days, and subsequent zero-order release kinetics for up to a month.

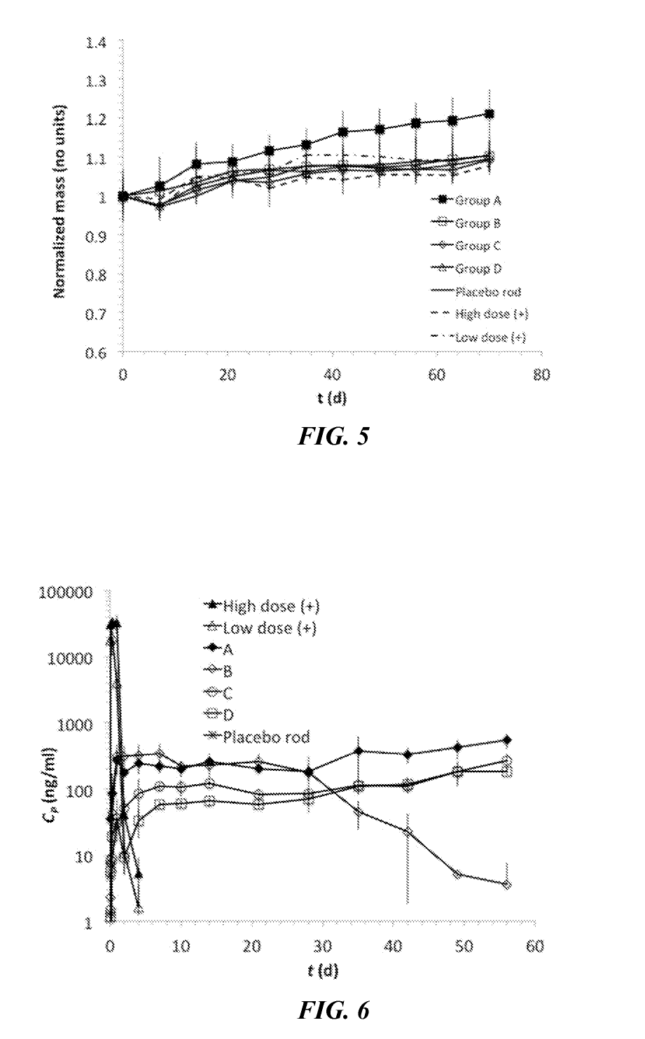

A follow up in vivo pharmacokinetic study was conducted on female Sprague-Dawley rats. There was no observable immune response or injection site issues related to the test articles throughout the in vivo study indicating that the silk rods were highly biocompatible. Furthermore, time evolution of the normalized body mass data collected over the whole duration of the study (FIG. 5) indicated no significant difference between any of the study groups. Table 1 summarizes silk reservoir rod dimensions and effective anastrozole load values for the test articles that were implanted subcutaneously. High (m_{A}=14 mg) and low dose (m_{A}=5.8 mg) anastrozole positive controls were injected subcutaneously as 1 ml solutions (water:ethanol, 7: 3 v/v).

TABLE 1
Silk rod dimensions and effective anastrozole load values
for in vivo and in vitro pharmacokinetic studies.

| | | m_{A}′ (M ± SD, mg/mm) |
|---|---|---|
| Group | dᵢ, dₒ, l (mm) | in vivo, in vitro |
| A | 1.5/2.0/20 | 1.4 ± 0.1, 1.3 ± 0.2 |
| B | 1.5/2.0/20 | 0.6 ± 0.1, 0.6(n = 1) |
| C | 1.0/2.0/20 | 0.8 ± 0.0, 0.7(n = 1) |
| D | 1.5/3.5/20 | 0.9 ± 0.1, 1.3 ± 0.1 |
| Placebo | 1.5/2.0/20 | 0.0 ± 0.0, NA |

FIG. 6 shows time evolution of plasma anastrozole concentration after the single administration per rat of high or low dose anastrozole positive control solution, silk-anastrozole reservoir rod (groups A to D) or placebo rod (n=3 for all groups). For both high and low dose anastrozole solution injection groups, plasma concentrations peaked at 6 h and rapidly declined to background levels within 96 h with an apparent terminal elimination half-life of approximately 6 h. On the other hand, all silk-anastrozole reservoir rod groups (groups A-D) showed essentially constant plasma concentrations for at least the first 28 days of the in vivo PK study. The plasma concentration was below the LC-MS/MS quantitation limit (LOQ≈0.6 ng/ml) for the placebo silk rod group. After the 28 days of release, the plasma concentration for group B gradually declined to the baseline value, presumably due to complete release of anastrozole, in good agreement with the in vitro data collected under the same rod dimensions and similar m_{A} values (FIG. 4). On the other hand, a gradual increase was observed in plasma concentrations for groups A, C and D after the first month, which could be attributed to anastrozole accumulation or silk biodegradation.

In vitro-In vivo Correlation (IVIVC):

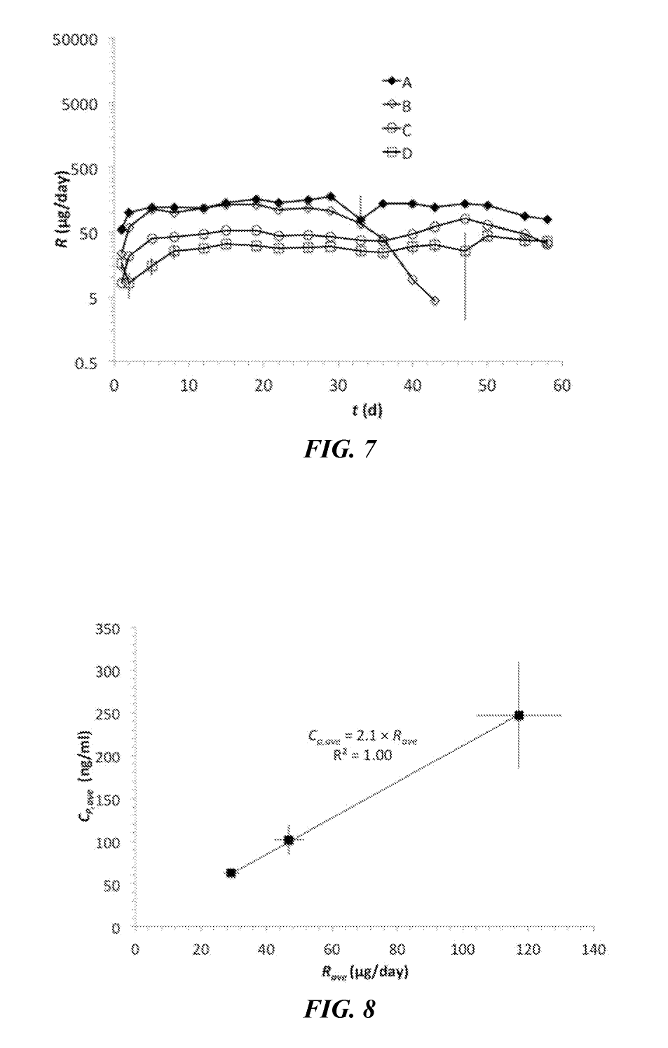

A parallel in vitro release assay was also conducted for groups A to D in Table 1. FIG. 7 shows the time evolution of in vitro daily anastrozole release rates. Essentially zero-order, sustained release kinetics were observed for all groups for the first 29 days. After 29 days, the daily release rate rapidly declined for group B (due to complete anastrozole release), while the release rate for groups A, C and D remained essentially constant up to 60 days, in contrast with the plasma concentration increase for in vivo samples for the latter. The increase in plasma concentration after the first month could be attributed to anastrozole accumulation or silk rod biodegradation. We hypothesize that finite accumulation of anastrozole due to its relatively long reported termination half-life (AstraZeneca Canada, Inc., ARIMIDEX™, Product Monograph, 2011) could lead to the observed increase in the plasma concentration. Such an accumulation effect would not be detectable in the in vitro dissolution system where the release medium was exchanged completely for each sampling. Alternatively, the increase in the plasma concentration may be due to differences between in vitro and in vivo silk degradation. For example, prior studies on silk matrices have shown that silk fibroin materials could biodegrade mainly through the action of proteolytic enzymes in vivo that are absent in the current in vitro dissolution system (Altman et al., Biomaterials, 2003, 24, 401). Such enzymatic biodegradation of silk rods could lead to a decrease in the effective film thickness and increase the in vivo release rate and the apparent plasma concentration of anastrozole.

FIG. 8 shows the dependence of average in vivo anastrozole plasma concentration between days 7 and 28, C_{p,ave} on the average in vitro daily release rate between days 8 and 29, Rₐᵥₑ for the study groups in table 1. A strict control over the release rate and subsequently the plasma concentration can be achieved simply by varying the rod dimensions according to Table 1. Furthermore, a strong IVIVC was observed with a simple empirical formula:

$$\overline{{C}_{p}} ( \frac{ng}{ml} ) = 2.1 \times R ( \frac{μ g}{d} ) \qquad [ 6 ]$$

Release Mechanism:

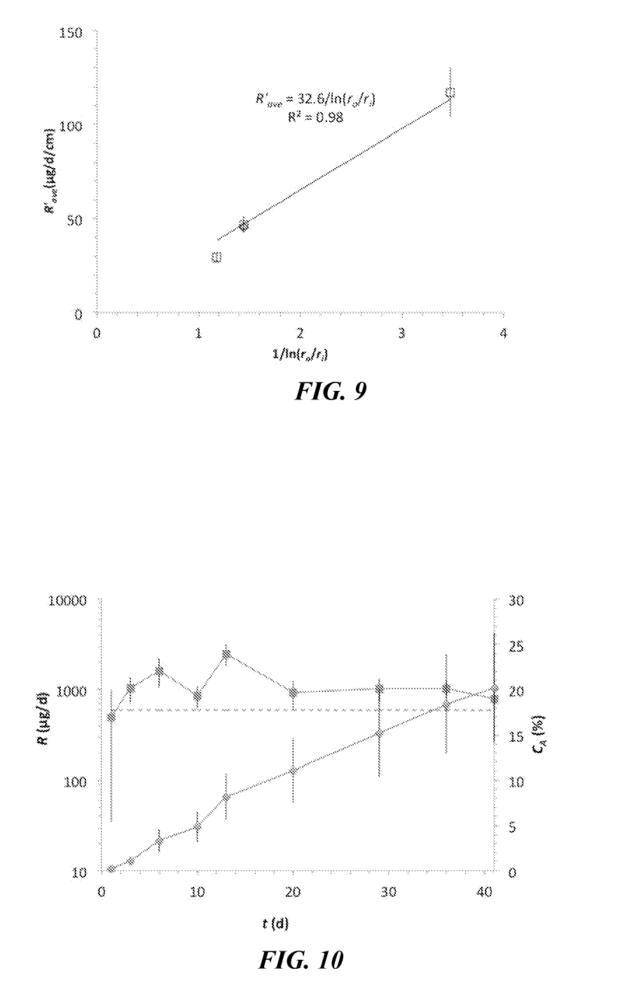

The average in vitro daily release rate, R values increased linearly with reciprocal ln

$$( \frac{{r}_{o}}{{r}_{i}} ) ,$$

where rₒ anu rᵢ are me outer and inner rod radii, respectively (FIG. 9). This dependence of release kinetics on rod dimensions is in good agreement with diffusion-limited, apparent equilibrium release kinetics from a cylindrical reservoir. Furthermore, there was no significant difference between anastrozole release rate from reservoir rods with

$${[ ln ( \frac{{r}_{o}}{{r}_{i}} ) ]}^{- 1} = 1.44$$

and different effective length, lₑ values of 10 or 30 mm. Therefore, the effective steady state diffusion coefficient, Dₑ of anastrozole from silk reservoir rods can be calculated using (Crank, Mathematics of Diffusion, Oxford: Clarendon Press, 1979)

$${D}_{e} = \frac{R}{2 π {l}_{e} {K}_{d} {C}_{s}} ln ( \frac{{r}_{o}}{{r}_{i}} ) \qquad [ 7 ]$$

where R is anastrozole release rate, lₑ is the effective rod length, K_{d} is the partition coefficient of anastrozole between silk and water, (≈5), Cₛ is the aqueous solubility of anastrozole in water (5 mg/ml), rₒ and rᵢ are the outer and inner rod radii. Using equation 6, we obtain a Dₑ value of 2.4×10⁻⁸ cm²/s for anastrozole diffusion in silk reservoir rods. A free hydrodynamic diffusion coefficient for anastrozole can be estimated as Dₒ≈5×10⁻⁶ cm²/s using Stokes-Einstein equation, assuming a spherical particle shape:

$${D}_{0} = \frac{kT}{6 π r η} \qquad [ 8 ]$$

where k is the Boltzmann constant, T is the absolute temperature, r is the hydrodynamic radius (estimated as ≈0.4 nm, assuming a spherical shape and a specific volume of ≈0.7 g/cm³) and η is the viscosity of water. Therefore Dₑ/Dₒ≈200, indicating possible physical interactions between anastrozole and silk films and/or a size exclusion effect (due to a correlation length(s) in a silk film that is comparable to the hydrodynamic radius of anastrozole). Hydrophobic forces may be the dominant physical interaction between silk (anionic with pI≈4) and moderately lipophilic anastrozole since the latter is not charged at neutral pH. However, the exact origins of possible physical interactions between anastrozole and silk should be studied further. To address a possible size exclusion effect, we need to consider the relative size of anastrozole and possible correlation lengths in a dense silk network, such as silk films. The hydrodynamic radius of anastrozole can be estimated as ≈0.4 nm, while silk fibroin is a high molecular weight protein (m≈350 kDa) with a hydrodynamic radius of ≈10 nm (Nagarkar et al. Physical Chemistry Chemical Physics, 2010, 12, 3834). On the other hand, one published SAXS report on low density, silk fibroin networks suggests the possibility of multiple correlation lengths (Nagarkar et al. Physical Chemistry Chemical Physics, 2010, 12, 3834). At length scales much larger than the hydrodynamic size of anastrozole, from that of the single fibroin molecule (10 nm) up to the correlation length of the network (>100 nm), a fractal dimension of D_{f}≈2.1 was observed that indicate a branched network, while at nanometer to sub-nanometer length scales, D_{f}≈1. This implies that at length scales smaller than its hydrodynamic radius, silk fibroin molecule may organize into extended, essentially one-dimensional, rod-like nanostructures, forming a low-density mesh. (Nagarkar et al. Physical Chemistry Chemical Physics, 2010, 12, 3834). Therefore, size-exclusion effect may be operational for anastrozole diffusion in dense silk films in the nanometer to sub-nanometer scale.

Controlling the Administration Frequency: A target daily in vitro anastrozole release rate of R_{T}≈600 μg/day was calculated assuming one-compartmental continuous infusion at steady state (Eq. 3) to attain the same range of clinical steady state plasma levels as that observed for the currently marketed 1 mg/day formulation. It should be emphasized that this calculation assumes no in vivo anastrozole accumulation due to sustained zero-order release from silk reservoir rods and as such may overestimate the required daily release rate since the present in vivo data suggests possible anastrozole accumulation (FIG. 6). Using equation 7, a high target release level (R_{T}≈600 μg/day), and currently achievable effective anastrozole load, m_{A}′ values, we can estimate reservoir rod dimensions that enable zero-order sustained release at the target level for different durations, which would translate into longer inter-administration duration of these formulations. Table 2 provides some exemplary silk reservoir rod dimensions for 1-12 month sustained delivery of anastrozole at the target level.

TABLE 2
Examplary silk-anastrozole reservoir rod dimensions (lₑ, effective
length, dₒ, outer diameter, Δr: silk film thickness) for an estimated
sustained delivery duration, t between one to twelve months

| t (months) | lₑ (mm) | dₒ (mm) | Δr (mm) |
|---|---|---|---|
| 1 | 10 | 1.93 | 0.09 |
| 3 | 30 | 2.06 | 0.15 |
| 6 | 40 | 2.66 | 0.26 |
| 9 | 40 | 3.26 | 0.32 |
| 12 | 37 | 3.85 | 0.35 |

A 41-day in vitro pilot release assay on silk-anastrozole rods with approximate dimensions for 360-day sustained delivery at the target level (lₑ, dₒ, Δr (mm)=36, 3.87, 0.35) gave an average in vitro daily release rate of 964±262 μg/day, and an expected sustained release duration of approximately 8 months (FIG. 10). These results suggest that it is possible to obtain sustained anastrozole delivery at or above the target release rate for over 6 months using silk reservoir rods.

Rod Biodegradation:

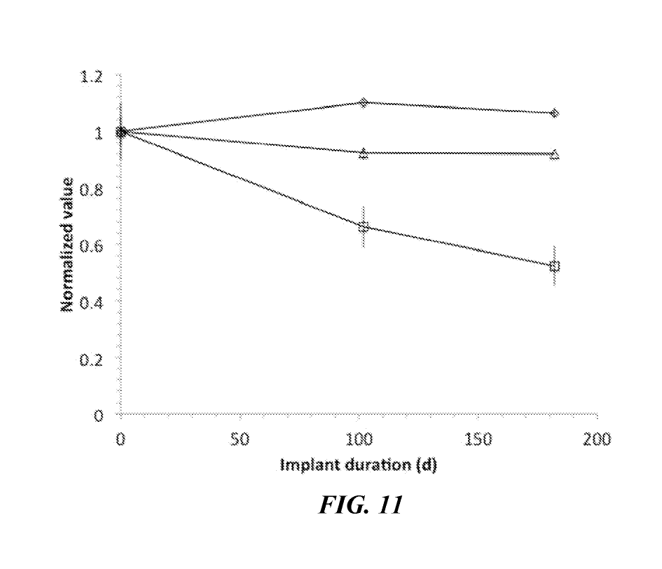

FIG. 11 shows time evolution of silk fibroin rod dry mass, β-sheet content measured by FT-IR spectroscopy and apparent mass averaged molecular weight measured by SEC as a function of implant duration in rats (n=3, values were normalized to pre-implant values). Normalized apparent mass averaged molecular weight values decreased gradually (66±7% and 52±7% at 102 and 182 days, respectively). In contrast, normalized dry mass values (92±1% and 92±2% at 102 and 182 days, respectively) and normalized β-sheet content values (110±1% and 106±2% at 102 and 182 days, respectively) remained essentially constant. In brief, silk fibroin reservoir rods sustained their overall structural integrity after 6 months of implantation in rats, providing sufficient time for several-month long, zero-order sustained delivery applications. The gradual decrease in apparent silk fibroin molecular weight over 182 days suggests that the rods may biodegrade completely over longer durations with a favorable biodegradation profile for controlled, sustained delivery applications.

A silk-protein based, reservoir rod was developed for zero-order and long-term sustained drug delivery applications. Silk reservoir rod formulations were processed in three steps. First, a regenerated silk fibroin solution, rich in random-coil content was transformed into a tubular silk film with desirable dimensions from injectable to implant size range, uniform film morphology and a structure rich in silk II, β-sheet content via “film-spinning.” Second, the drug powder was loaded into swollen silk tubes followed by tube end clamping. Last, clamped silk tube ends were sealed completely via dip coating. Anastrozole, an FDA approved active ingredient for the treatment of breast cancer, was used as a model drug to investigate viability of the silk reservoir rod technology for sustained delivery. In vitro and in vivo (female Sprague-Dawley rats) pharmacokinetic data analyzed via liquid chromatography-tandem mass spectroscopy indicated zero-order release for months. In vitro anastrozole release rate could be controlled simply by varying silk rod dimensions, while in vivo results highlighted a strong in vitro-in vivo correlation and silk rod biocompatibility. Silk film swelling and zero-order anastrozole release kinetics indicated practically immediate film hydration and formation of a linear anastrozole concentration gradient along the silk film thickness. The dependence of anastrozole release rate on the overall rod dimensions was in good agreement with essentially diffusion-controlled, zero-order sustained release from a reservoir cylindrical geometry. Overall, silk reservoir rod may be a viable candidate for sustained delivery of breast cancer therapeutics.

Decreasing the frequency of drug administration is a major target in pharmaceutical research. Benefits of decreased administration frequency include improved patient compliance, convenience and overall quality of life. The work described herein provides film-spun silk-based materials as a versatile material platform for sustained delivery. Near zero-order, sustained delivery obtained from anastrozole for several months show that the film-spun silk-based materials are viable for breast cancer therapy.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A sustained delivery composition, the composition comprising
(i) a silk matrix comprising a lumen; and
(ii) an anti-cancer agent;
wherein the anti-cancer agent is in the lumen; and two ends of the lumen are closed to retain the anti-cancer agent within the lumen.

2. The composition of claim 1, wherein the silk matrix is a cylindrical shape.

3. The composition of claim 1, wherein the silk matrix has a length of from about 1 mm to about 10 cm.

4. The composition of claim 3, wherein the silk matrix is has a length of about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm.

5. The composition of claim 1, wherein the silk matrix has a wall thickness of from about 50 μm to about 5 mm.

6. The composition of claim 5 wherein the silk matrix has a wall thickness of about 0.09 mm, about 0.10 mm, about 0.15 mm, about 0.21 mm, about 0.24 mm, about 0.25 mm, about 0.26 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, or about 4 mm.

7. The composition of claim 1 wherein the silk matrix has a diameter from about from about 0.5 mm to about 10 mm.

8. The composition of claim 7 wherein the silk matrix has a diameter of about 1 mm, about 1.25 mm, about 1.5 mm, about 1.75 mm, about 1.93 mm, about 1.95 mm, about 2 mm, about 2.06 mm, about 2.17 mm, about 2.25 mm, about 2.43 mm, about 2.5 mm, about 2.66 mm, about 2.75 mm, about 3 mm, about 3.25 mm, about 3.5 mm, about 3.75 mm, about 4 mm, about 4.25 mm, about 4.5 mm, about 4.75 mm, or about 5 mm.

9. The composition of claim 1, wherein the lumen has a diameter from about from about 100 nm to about 10 mm.

10. The composition of claim 9, wherein the lumen has a diameter of about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm. about 3 mm, about 3.25 mm, or about 3.5 mm.

11. The composition of claim 1, wherein the lumen has a length of from about 1 mm to about 10 cm.

12. The composition of claim 11, wherein the lumen has a length of about 5 mm, about 7.5 mm, about 10 mm, about 12.5 mm, about 15 mm, about 17.5 mm, about 20 mm, about 22.5 mm, about 25 mm, about 27.5 mm, about 30 mm, about 32.5 mm, about 35 mm, about 37.5 mm, about 40 mm, about 42.5 mm, about 45 mm, about 47.5 mm, or about 50 mm.

13. The composition of claim 1, wherein silk fibroin in the silk matrix comprises silk II beta-sheet crystallinity of at least 5%.

14. The composition of claim 13, wherein silk fibroin in the silk matrix comprises silk II beta-sheet crystallinity of about 47%.

15. The composition of claim 1, wherein the anti-cancer agent is an anti-breast cancer agent.

16. The composition of claim 1, wherein the anti-cancer agent is selected from the group consisting of adrenal corticosteroid inhibitors, alkylating agents, androgens and anabolic steroids, antibiotics/antineoplastics, antimetabolites, aromatase inhibitors, EGFR inhibitors and HER2 inhibitors, estrogen receptor antagonists, estrogens, HER2 inhibitors, immunosuppressants, mitotic inhibitors, mTOR inhibitors, selective immunosuppressants, selective estrogen receptor modulators, and VEGF/VEGFR inhibitors, and any combinations thereof

17. The composition of claim 1, wherein the anti-cancer agent is anastrozole.

18. The composition of claim 1, wherein the composition comprises from about 0.01% to about 95%(w/w) of the anti-cancer agent.

19. The composition of claim 1, wherein the composition comprises from about 0.5 mg to about 2.5 mg of the anti-cancer agent per mm of length of the silk matrix or the lumen.

20. The composition of claim 19, wherein the composition comprises about 0.5 mg, about 0.6 mg, about 0.7 mg, about 0.8 mg, about 0.9 mg, about 1 mg, about 1.1 mg, about 1.2 mg, about 1.3 mg, about 1.4 mg, or about 1.5 mg of the anti-cancer agent per mm of length of the silk matrix or the lumen.

21. The composition of claim 1, wherein the silk matrix further comprises a biocompatible polymer.

22. The composition of claim 1, wherein the composition is implantable or injectable.

23. The composition of claim 1, wherein the silk matrix has the dimensions:
(i) a length of about 10 mm, a lumen of diameter about 1.5 mm, and an outer diameter of about 2.0 mm;
(ii) a length of about 20 mm, a lumen diameter of about 1.5 mm, and an outer diameter about 2.0 mm;
(iii) a length of about 20 mm, a lumen diameter of about 1.0 mm, and an outer diameter about 2.0 mm;
(iv) a length of about 20 mm, a lumen diameter of about 1.5 mm, and an outer diameter about 3.5 mm;
(v) a length of about 46 mm, a lumen diameter of about 3.2 mm, and an outer diameter about 3.9 mm; or
(vi) a length of about 36 mm, a lumen diameter of about 3.9 mm, and an outer diameter about 3.5 mm.

24. The composition of claim 1, wherein the composition comprises:
(i) the silk matrix having a length of about 10 mm, a lumen of diameter about 1.5 mm, and an outer diameter of about 2.0 mm; and about 1.3 mg or about 1.4 mg of the anti-cancer agent per mm of length of the silk matrix;
(ii) the silk matrix having a length of about 20 mm, a lumen diameter of about 1.5 mm, and an outer diameter about 2.0 mm; and about 0.6 mg of the anti-cancer agent per mm of length of the silk matrix;
(iii) the silk matrix having a length of about 20 mm, a lumen diameter of about 1.0 mm, and an outer diameter about 2.0 mm; and about 0.8 mg or about 0.7 mg of the anti-cancer agent per mm of length of the silk matrix;
(iv) the silk matrix having a length of about 20 mm, a lumen diameter of about 1.5 mm, and an outer diameter about 3.5 mm; about 0.9 mg or about 1.3 mg of the anti-cancer agent per mm of length of the silk matrix; or
(v) the silk matrix having a length of about 46 mm, a lumen diameter of about 3.2 mm, and an outer diameter about 3.9 mm; and about 6 mg of the anti-cancer agent per mm of length of the silk matrix.

25. The composition of claim 1, wherein the silk matrix has the dimensions:
(i) a lumen length of about 10 mm, a lumen diameter of about 1.75 mm, and an outer diameter of about 1.93 mm;
(ii) a lumen length of about 20 mm, a lumen diameter of about 1.75 mm, and an outer diameter of about 1.95 mm;
(iii) a lumen length of about 30 mm, a lumen diameter of about 1.76 mm, and an outer diameter of about 2.06 mm, and wall thickness of about 0.15 mm;
(iv) a lumen length of about 40 mm, a lumen diameter of about 1.75 mm, and an outer diameter of about 2.17 mm;
(v) a lumen length of about 40 mm, a lumen diameter of about 1.95 mm, and an outer diameter of about 2.43 mm;
(vi) a lumen length of about 40 mm, a lumen diameter of about 2.14 mm, and an outer diameter of about 2.66 mm;
(vii) a lumen length of about 46 mm, a lumen diameter of about 3.2 mm, and an outer diameter of about 3.9 mm; or
(viii) a lumen length of about 36 mm, a lumen diameter of about 3.5 mm, and an outer diameter of about 3.9 mm.

26. The composition of claim 1, wherein the composition provides sustain release of the anti-cancer agent over a period of at least about a week.

27. The composition of claim 1, wherein anti-cancer agent is released from the composition at a rate of from about 1 μg/day to about 10 mg/day.

28. The composition of claim 27, wherein the anti-cancer agent is released from the silk matrix at a rate of about 600 to about 1000 μg/day.

29. The composition of claim 1, wherein the anti-cancer agent has duration of therapeutic effect which is at least one day longer relative to duration of therapeutic effect in the absence of the silk matrix.

30. A pharmaceutical composition comprising a sustained delivery composition of claim 1 and a pharmaceutically acceptable carrier.

31. A method for treating cancer in a subject, the method comprising administering to a subject in need thereof a composition of claim 1.

32. The method of claim 31, wherein administration frequency of the composition is less than when the same amount of the anti-cancer agent is administered in the absence of the silk matrix.

33. The method of claim 32, wherein the administration frequency is reduced by a factor of ½ relative to when the anti-cancer agent is administered in the absence of the silk matrix.

34. The method of claim 31, wherein said administration is no more than once a month, no more than once every two week, no more than once every three weeks, no more than once a month, no more than once every two months, no more than once every four months or no more once every six months.

35. A drug delivery device comprising the composition of claim 1.

36. The drug delivery device of claim 35, wherein the drug delivery device is a syringe with an injection needle.

37. The drug delivery device of claim 36, wherein the device is an implant.

38. A kit comprising a composition of claim 1, or a drug delivery device of claim 35.

39. The kit of claim 38, further comprising at least a syringe and an injection needle.

40. The kit of claim 38, further comprising an anesthetic.

41. The kit of claim 38, further comprising an antiseptic agent.

42. The kit of claim 38, further comprising instruction for use.

43. A method of preparing a sustained delivery composition of claim 1, the method comprising:
(i) forming a silk tube, wherein forming the silk tube comprises:
a. delivering, with an applicator, a silk solution onto a support structure, wherein the support structure is an elongated structure with a longitudinal axis, and wherein the support structure is reciprocated horizontally while being rotated along its longitudinal axis to form a silk coating thereon;
b. heating the silk coating, while rotating the wire, to form a silk film; and
c. optionally repeating the delivering and heating steps to form one or more coatings of silk film thereon;
(ii) inducing a conformational change in the silk coating;
(iii) optionally hydrating the silk tube;
(iv) loading the silk tube with an anti-cancer agent;
(v) closing ends of the silk tube such that the therapeutic agent is sealed therein.
